SEQUENCE LISTING (1 sequence)

SEQ ID NO: 1 - protein, 8 aa, chicken
SIINFEKL

(19) United States
(12) Patent Application Publication
(10) Pub. No.: US 2013/0189302 A1
(43) Pub. Date: Jul. 25, 2013

(54) IMMUNOTHERAPEUTIC METHOD USING ARTIFICIAL ADJUVANT VECTOR CELLS THAT CO-EXPRESS CD1D AND TARGET ANTIGEN

(71) Applicant: Riken, Wako-shi (JP)

(72) Inventors: Shin-ichiro FUJII, Yokohama-shi (JP); Kanako SHIMIZU, Yokohama-shi (JP)

(73) Assignee: RIKEN, Wako-shi (JP)

(21) Appl. No.: 13/834,096

(22) Filed: Mar. 15, 2013

Related U.S. Application Data
(63) Continuation-in-part of application No. 13/131,299, filed on Jul. 27, 2011.
Application No. 13/131,299 is a continuation-in-part of application No. 12/280,305, filed on Oct. 29, 2008.

(30) Foreign Application Priority Data
Feb. 22, 2006 (JP) 2006-045193
Nov. 28, 2008 (JP) 2008-305639

(51) Int. Cl. C12N 15/85; A61K 39/12; A61K 39/00

(52) U.S. Cl. CPC C12N 15/85; A61K 39/0011; A61K 39/12
USPC 424/204.1; 435/375; 435/325; 424/278.1; 424/277.1

(57) ABSTRACT

Provided is immunotherapy of cancer or infection utilizing activation of dendritic cell (DC) by innate immunity, namely, a method of preparing an artificial adjuvant vector cell co-expressing a target antigen and CD1d and having an ability to activate immunity against the target antigen, comprising treating the target antigen and CD1d co-expressing cell with a CD1d ligand in a culture medium.

### CROSS-REFERENCE TO RELATED APPLICATIONS

This patent application is (a) a continuation-in-part of copending U.S. patent application Ser. No. 13/131,299, filed Jul. 27, 2011, which is the U.S. national phase of International Patent Application No. PCT/JP2009/070061, filed Nov. 27, 2009, which claims the benefit of Japanese Patent Application No. 2008-305639, filed Nov. 28, 2008, and (b) a continuation-in-part of copending U.S. patent application Ser. No. 12/280,305, filed Oct. 29, 2008, which is the U.S. national phase of International Patent Application No. PCT/JP2007/053209, filed Feb. 21, 2007, which claims the benefit of Japanese Patent Application No. 2006-045193, filed Feb. 22, 2006, and all of which applications are incorporated by reference in their entireties herein.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 650 Byte ASCII (Text) file named “712050SequenceListing.txt” created on Mar. 15, 2013.

### BACKGROUND OF THE INVENTION

NKT cells are cells of a lymphocyte lineage simultaneously having the characteristics of both T cells and NK cells, which are activated by an antigen present on MHC class I-like molecule CD1d, and have antitumor activity. Examples of the antigen (CD1d ligand) present on CD1d, which strongly activates NKT cells, include glycolipid α-galactosylceramide (α-GalCer). Primary NKT cells are stimulated by antigen presenting cells (APC) loaded with an antigen such as α-GalCer and the like, and can differentiate and proliferate. Examples of such APC include macrophages, immature or mature dendritic cells (DC) and the like. Of these, the present inventors previously reported that mature DC pulsed with α-GalCer strongly activates NKT cells.

The NKT cells activated by ex vivo mature DCs loaded with α-GalCer have been shown to be a direct effect on tumors and also to generate an NK-mediated indirect antitumor effect. (see, e.g., Fujii et al., Nature Immunology, 3: 867-874 (2002); Fujii et al., Journal of Immunological Methods, 272: 147-159 (2003)). This method is applicable to various antitumor immunotherapies of leukemia and the like.

In immunotherapy using NKT cells, co-administration of a CD1d ligand and a tumor antigen or dendritic cell affords induction of antigen-specific T cell immunity as an adjuvant effect (see, e.g., Fujii et al., Nature Immunology, 3: 867-874 (2002); Fujii et al., The Journal of Experimental Medicine, 198: 267-279 (2003); Fujii et al., The Journal of Experimental Medicine, 199: 1607-1618 (2004); Metelitsa et al., The Journal of Immunology, 167: 3114-3122 (2001); Fais et al., International Journal of Cancer, 109: 402-411 (2004); Hermans et al., The Journal of Immunology, 171: 5140-5147 (2003); and Silk et al., The Journal of Clinical Investigation, 144: 1800-1811 (2004))).

On the other hand, in mouse models, it was shown that the co-administration approach is limited as a method in terms of timing and the number of cells because an injection of tumor antigen after the CD1d ligand did not generate T cell efficiently. Also, for many antigens, this method required the co-injection of irradiated tumor cells and NKT ligand, which resulted in death of mice due to the embolism of tumor cells in the lung (only particular cell lines, such as the J558 cell line, could be used) (Liu et al., The Journal of Experimental Medicine, 202: 1507-1516 (2005)).

An attempt to induce an antigen-specific immunotherapy by transfecting an mRNA derived from a tumor antigen into a dendritic cell has already been established and used for clinical applications (see, e.g., J. Exp. Med. 184: 465-472 (1996); Nat. Med., 2: 1122-1128 (1996); Nat. Med., 6: 1011-1017 (2000); J. Clin. Invest., 109: 409-417 (2002); J. Immunol., 174: 3798-3807 (2005); Br. J. Cancer, 93: 749-756 (2005); and Cancer Gene Ther., 13: 905-918 (2006)). However, the problem of this method is that mRNA transfection efficiency and expression level of tumor antigen in the dendritic cells are still weak, and an attempt has been made to improve the treatment effect by the concurrent use of an adjuvant.

### BRIEF SUMMARY OF THE INVENTION

The present invention provides a method of preparing an artificial adjuvant vector cell (aAVC) co-expressing a target antigen and CD1d (i.e., a target antigen and CD1d co-expressing cell) and having an ability to activate immunity against the target antigen, comprising treating (e.g., by pulsing or loading) the target antigen and CD1d co-expressing cell with a CD1d ligand in a culture medium, as well as an aAVC obtained by the method and a composition comprising the aAVC. An aAVC of the present invention is a cell that co-expresses a target antigen and CD1d, loaded with CD1d ligand. The target antigen and CD1d co-expressing cell of the present invention may be the cell produced by the step of (a) transfecting a nucleic acid (e.g., mRNA or vector) encoding a target antigen or CD1d into a cell expressing the target antigen or CD1d, or (b) transfecting 1 or 2 molecular species of a nucleic acid (e.g., mRNA or vector) encoding a target antigen and CD1d into a cell.

In particular, the present invention provides an aAVC co-expressing a target antigen and CD1d, which is treated to co-express a target antigen and CD1d, or enhance expression of a target antigen and/or CD1d. The base cell of the aAVC (the cell prior to transfection) is selected from (i) a CD1d-expressing cell, (ii) a target antigen-expressing cell, or (iii) a cell having no expression of a target antigen and CD1d. The CD1d-expressing cell includes (i-i) a cell naturally expressing CD1d and (i-ii) a cell previously transfected with a nucleic acid (e.g., vector) encoding CD1d before the step of (a) or (b) (CD1d transfectant). The target antigen-expressing cell includes (ii-i) a cell naturally expressing the target antigen and (ii-ii) a cell previously transfected with a nucleic acid (e.g., vector) encoding the target antigen before the step of (a) or (b) (target antigen transfectant). The target antigen and CD1d co-expressing cell of the present invention also may be the cell enhanced by the expression of the target antigen and/or CD1d by the step of (a) or (b) when the base cell of the aAVC has the expression of the target antigen and/or CD1d.

The present invention also provides a kit comprising any of (1) to (8): (1) a combination of (1-1) a CD1d-expressing cell, and (1-2) a construct for in vitro transcription of a nucleic acid encoding the target antigen; (2) a combination of (2-1) a CD1d-expressing cell, and (2-2) a nucleic acid encoding the target antigen; (3) a combination of (3-1) a construct for in vitro transcription of a nucleic acid encoding CD1d, (3-2) a construct for in vitro transcription of an mRNA encoding the target antigen, and (3-3) a cell that is allogeneic to a target in need of immunity induction; (4) a combination of (4-1) a construct for in vitro transcription of a nucleic acid encoding CD1d and a nucleic acid encoding the target antigen, and (4-2) a cell that is allogeneic to a target in need of immunity induction; (5) a combination of (5-1) a construct for in vitro transcription of a nucleic acid encoding CD1d, (5-2) a nucleic acid encoding the target antigen, and (5-3) a cell that is allogeneic to a target in need of immunity induction; (6) a combination of (6-1) a nucleic acid encoding CD1d and the target antigen, (6-2) a construct for in vitro transcription of a nucleic acid encoding the target antigen, and (6-3) a cell that is allogeneic to a target in need of immunity induction; (7) a combination of (7-1) a nucleic acid encoding CD1d and the target antigen, (7-2) a nucleic acid encoding the target antigen, and (7-3) a cell that is allogeneic to a target in need of immunity induction; and (8) a combination of (8-1) a nucleic acid encoding CD1d and the target antigen, and (8-2) a cell that is allogeneic to a target in need of immunity induction.

Additionally, the present invention provides a method of inducing immunity comprising administering an effective amount of the aAVC to a subject in need thereof, wherein the cell is syngeneic or allogeneic to the subject.

### BRIEF DESCRIPTION OF THE SEVERAL VIEWS OF THE DRAWING(S)

FIG. 1 shows the IFN-γ production amount of a mouse liver-derived mononuclear cell co-cultured with a tumor cell pulsed with α-GalCer. CD1dB16: CD1d expression-enhanced B16; CD1dEL4: CD1d expression-enhanced EL4; DC: dendritic cell; WT: wild-type; JαKO: Ja281 gene deficient mouse (Va14+NKT cell deficient mouse); −: not pulsed with α-GalCer; +: pulsed with α-GalCer.

FIG. 2 shows IFN-γ production amount in mouse immunized with a tumor cell pulsed with various concentrations of α-GalCer. Gal: α-GalCer; DC/G: dendritic cell pulsed with α-GalCer; B16/G: B16 pulsed with α-GalCer; CD1dB16/G: CD1d expression-enhanced B16 pulsed with α-GalCer.

FIG. 3 shows difference in antitumor immunity in lung metastasis models.

FIG. 4 shows antitumor immune response in mice immunized with tumor cells pulsed with α-GalCer. Tumor growth (mm²) is indicated on the y-axis and time lapse (days) after tumor inoculation is indicated on the x-axis.

FIG. 5 shows relative expression levels of CD1d mRNA among tumor cell lines, as measured by a real time RT-PCR method. The relative level of RNA expression (CD1d/18S) is indicated on the y-axis for the tumor cell lines with and without CD1d transfection (x-axis).

FIG. 6 shows relative expression levels of CD1d protein among tumor cell lines, as measured by flow cytometry. DC: dendritic cell; CD1dB16: CD1d expression-enhanced B16; CD1dEL4: CD1d expression-enhanced EL4; isotype: ratIgG2b.

FIG. 7 shows a killing effect provided by a liver-derived mononuclear cells on tumor cells pulsed or not pulsed with CD1d ligand. Percent cytoxicity is indicated on the y-axis and the E/T ratio is indicated on the x-axis. E/T ratio: effecter (E)/target (T) ratio, where liver-derived mononuclear cell containing NKT cell and NK cell corresponds to E, and a tumor cell corresponds to T.

FIG. 8 shows the measurement results of CFSE uptake by dendritic cell using a flow cytometer.

FIG. 9 is a schematic diagram showing the process of in vitro preparation of mRNA for the expression of a protein in a cell from a full-length cDNA encoding the protein cloned into a vector.

FIG. 10A shows the results of FACS analysis of EGFP expression levels of B16 (upper) or NIH3T3 (lower) cells transfected with 5 μg of EGFP mRNA, wherein 53.5 and 68.3 in the graphs respectively show the percentages of GFP positive cells

FIG. 10B shows the OVA expression levels (y-axis; ng/ml) as determined by ELISA of B16 melanoma cells after incubation for the time shown by 1° on the x-axis, transfection with 5 μg of OVA mRNA, and lapse of the time shown by 2°.

FIG. 10C shows the OVA expression levels (y-axis; ng/ml) as determined by ELISA of NIH3T3 cells after incubation for the time shown by 1° on the x-axis, transfection with 5 μg of OVA mRNA, and lapse of the time shown by 2°.

FIG. 10D shows the OVA expression levels (y-axis; ng/ml) as determined by ELISA of EL4 cells after incubation for the time shown by 1° on the x-axis, transfection with 5 μg of OVA mRNA, and lapse of the time shown by 2°.

FIG. 11A shows the CD1d-expression level (y-axis) of each cell shown on the x-axis, which is quantified by real-time PCR and indicated by a relative value to rRNA.

FIG. 11B shows CD1d and EGFP expression levels (left) and the results of FACS analysis of CD1d expression level of each cell shown on the y-axis (right).

FIG. 11C shows the OVA secretion amount (y-axis; ng/ml) of each cell shown on the x-axis, which was transfected with OVA mRNA, as measured by ELISA 4 hr later.

FIG. 11D shows the IFN-γ secretion amount (y-axis; ng/ml) as measured by ELISA in the supernatant obtained by co-culture of CD8⁺ T cells transfected with OVA (OT-I (OT-1 in the figure) cell) and each cell shown on the x-axis.

FIG. 11E shows the OT-I cell numbers from mouse spleen (y-axis), which was obtained by administering OT-I cells to mice, immunizing the mice 24 hr later with an OVA mRNA transfectant loaded or not loaded with α-GalCer, and measuring the number 3 days later.

FIG. 12A shows the results of FACS analysis of the expression level of each protein shown on the x-axis in the mouse spleen cell immunized with each cell described on the y-axis.

FIG. 12B shows the level as measured by ELISPOT assay of IFN-γ produced by spleen cell of mouse immunized with each cell described on the x-axis.

FIG. 12C shows difference in antitumor immunity in lung metastasis models by the lung image and the number of lung metastasis.

FIG. 13A shows the results of FACS analysis of the expression level of each protein shown on the x-axis in DC of mouse immunized with each cell described on the y-axis and shown for CD8a⁺ and CD8a⁻ DC subsets.

FIG. 13B shows CD70 expression levels of the mouse DC immunized with each cell described on the y-axis, which was analyzed by FACS 12 hr and 40 hr after immunization and shown for CD8a⁺ and CD8a⁻ DC subsets.

FIG. 13C shows CD8 and IL-12 expression levels of each cell by a flow cytometer.

FIG. 13D shows evaluation of OT-I cell growth in the mouse described in the y-axis, which was administered with CFSE-labeled OT-I cell, with or without immunization with CD1d^{hi}-NIH3T3/Gal-ova (CD1dNIH/Gal-ova).

FIG. 14A shows the amounts of CD8 and OVA peptides on the spleen cell surface of the mouse immunized with the cell described in the upper part of each graph.

FIG. 14B shows the amounts of CD8 and OVA peptides on the spleen cell surface of the mouse immunized with CD1d^{hi}-NIH3T3/Gal-ova cell and described in the upper part of each graph.

FIG. 14C shows the amounts of CD8 and OVA peptides on the spleen cell surface of the mouse immunized with the cell described in the upper part of each graph.

FIG. 14D shows the quantification results of IFN-γ secreted by co-culture of CD8⁺ cells of the mouse immunized with the cells described on the x-axis and CD11c+ pulsed with OVA peptide.

FIG. 15A shows the size of tumor in a mouse after immunization with the cell underlined in each graph, subcutaneous administration of EG7 (left) or EL4 (right) 2 weeks later, and lapse of the number of days shown on the x-axis.

FIG. 15B shows the size of tumor in a gene knockout mouse underlined in each graph after immunization with CD1d^{hi}-NIH3T3/Gal-ova, subcutaneous administration of EG7 2 weeks later, and lapse of the number of days shown on the x-axis.

FIG. 16A shows the expression level of trp2 in a cell shown by each lane number as measured by RT-PCR.

FIG. 16B shows the expression level of trp2 (y-axis) in each cell shown on the x-axis as quantified by real-time PCR.

FIG. 16C shows the size of tumor (y-axis; mm²) in a mouse after immunization with a pair of the cells described in the upper right and upper left of each graph, subcutaneous administration of the cell underlined in each graph 2 weeks later, and lapse of the number of days shown on the x-axis.

FIG. 17A shows the frequency of CD4⁺T cells and CD8⁺T cells among total CD3⁺ T cells in mice following two doses of aAVCs (low dose; 5×10⁶, high dose; 5×10⁷) as assessed by flow cytometry.

FIG. 17B shows representative data of iNKT cell frequency in dogs injected with a high dose of aAVC as assessed by flow cytometry.

FIG. 17C is a graph showing the number of IFN-γ producing cells in aAVCs-ova immunized dogs. Data shown are mean±S.E.M. of 3 dogs per each group. (*P<0.05, pre versus 1 week).

FIG. 17D is a graph showing serum canine IL-12 (pg/ml; y-axis) as measured by ELISA (R&D Systems) in aAVC-injected dogs at the indicated time points after immunization with aAVC-ova. Left panel is the representative data of high dose (▪) and low dose (◯) aAVC injected groups, and the right panel shows the mean±S.E.M. (The control is the data of six unimmunized dogs.)

FIG. 17E is a graph showing the number of IFN-γ producing cells. Seven days after immunization with aAVC-ova, T cell responses to OVA were evaluated by canine IFN-γ-ELISPOT. CD8⁺T cells were isolated from PBMC of immunized dogs using rat anti-dog CD8-PE (Serotec) and PE-magnetic beads (Miltenyi) and then restimulated with or without OVA protein-transfected canine DCs for 36 hours before the ELISPOT assay. Data shown are mean±S.E.M. of 3 dogs per each group. (*P<0.05, −OVA versus +OVA)

### DETAILED DESCRIPTION OF THE INVENTION

It has been considered desirable to use dendritic cells derived from a patient, in immunotherapy, since the autologous dendritic cell actually activates NK cells and CD8+T cells in the body.

However, the present inventors have found that a CD1d ligand pulsed (loaded)-cell which expresses a tumor antigen and CD1d induces a very strong immune response specific to the tumor antigen, particularly simultaneous activation of NK/NKT cells and T-cell immune response. The present inventors also have found that the CD1d ligand pulsed-cell can induce both natural immunity and acquired immunity against the target antigen, leading to immunological memory. In particular, the inventors found that when a CD1d ligand is presented by a CD1d-expressing tumor, strong in vivo activation of IFN-γ producing NKT cells and NK cells can be sustained, that the tumor cell is killed by the activation of NK/NKT cells, and the antigen of the tumor is captured and presented by the adjacent dendritic cell, such that the acquired immunity to the antigen can be later induced. Once the acquired immunity is induced, tumor immunity is input in the memory. In this way, a CD1d-expressing tumor cell pulsed with an antigen can induce natural immunity in the short run and acquired immunity in the long run.

Furthermore, the present inventors have unexpectedly found that allogeneic culture cells, which have been transfected with a nucleic acid (e.g., mRNA or vector) encoding a target antigen and/or CD1d and have been confirmed to have sufficient expression of the target antigen and CD1d, can exhibit an effect equivalent to use of the autologous cell. This means that a cell usable for immunotherapy can be ensured with substantially no limit, which is a considerable achievement for treatment.

The present inventors have made further studies and developed a “cell kit” for immunotherapy, using a CD1d ligand, nucleic acid (e.g., mRNA and/or vector) of a tumor or virus and an allogeneic antigen presenting cell for presenting a target antigen and CD1d.

The present invention provides a method of preparing an artificial adjuvant vector cell (aAVC) co-expressing a target antigen and CD1d and having an ability to activate immunity against the target antigen, comprising treating (e.g., by pulsing or loading) the target antigen and CD1d co-expressing cell with a CD1d ligand in a culture medium, as well as an aAVC obtained by the method and a composition comprising the aAVC.

In particular, the present invention provides an aAVC co-expressing a target antigen and CD1d, which is treated to co-express a target antigen and CD1d, or enhance expression of a target antigen and/or CD1d. The target antigen and CD1d co-expressing cell of the present invention may be the cell produced by the step of (a) transfecting a nucleic acid (e.g., mRNA or vector) encoding a target antigen or CD1d into a cell expressing the target antigen or CD1d, or (b) transfecting 1 or 2 molecular species of a nucleic acid (e.g., mRNA or vector) encoding a target antigen and CD1d into a cell. The base cell (i.e., the cell prior to transfection) of the aAVC is selected from: (i) a CD1d-expressing cell, (ii) a target antigen-expressing cell, or (iii) a cell having no expression of a target antigen and CD1d. The target antigen and CD1d co-expressing cell of the present invention also may be the cell enhanced by the expression of the target antigen and/or CD1d by the step of (a) or (b) when the base cell of aAVC has the expression of the target antigen and/or CD1d. The enhancement of expression of the target antigen and/or CD1d by transfection of nucleic acid can be enhanced to the degree that the treatment effect of the immunotherapy using the cell of the present invention is sufficiently increased.

(1. Cell)

The present invention provides a cell co-expressing a target antigen and CD1d, which is loaded with a CD1d ligand, particularly a cell co-expressing a target antigen and CD1d, which is obtained by the step of (a) transfecting a nucleic acid (e.g., mRNA or vector) encoding a target antigen or CD1d into a cell expressing the target antigen or CD1d or (b) transfecting one or two molecular species of a nucleic acid (e.g., mRNA or vector) encoding a target antigen and CD1d into a cell. Such co-expressing cell loaded with a CD1d ligand has an ability to activate immunity against the target antigen.

In the above-mentioned the step of (b), the “one molecular species of nucleic acid encoding a target antigen and CD1d” means both the target antigen and CD1d are encoded by one nucleic acid, and the “two molecular species of nucleic acid encoding a target antigen and CD1d” means each of the target antigen and CD1d is encoded by a separate nucleic acid. The nucleic acid encoding both the target antigen and CD1d are also referred to as “a nucleic acid (e.g., mRNA or vector) encoding the target antigen” or “a nucleic acid (e.g., mRNA or vector) encoding CD1d.”

In a first embodiment, a cell co-expressing a target antigen and CD1d is prepared by transfecting a CD1d-expressing cell with a nucleic acid (e.g., mRNA or vector) encoding the target antigen. The CD1d-expressing cell may be (i-i) a cell naturally expressing CD1d or (i-ii) a cell previously transfected with a nucleic acid (e.g., mRNA or vector) encoding CD1d (CD1d transfectant). The CD1d-expressing cell may be a syngeneic cell or allogeneic cell. Preferably the cell expressing CD1d is an allogeneic cell (in view of the large-scale production). The CD1d-expressing cell also may naturally or artificially express target antigen, or have no expression of the target antigen. When the cell naturally or artificially expresses the target antigen, the transfection of the cell with a nucleic acid (e.g., mRNA or vector) encoding a target antigen can be used to enhance the expression of the target antigen.

In a second embodiment, a cell co-expressing a target antigen and CD1d is prepared by transfecting a cell expressing the target antigen with a nucleic acid (e.g., mRNA or vector) encoding CD1d. The cell expressing the target antigen (target antigen-expressing cell) may be (ii-i) a cell naturally expressing the target antigen or (ii-ii) a cell previously transfected with a nucleic acid (e.g., vector) encoding the target antigen (target antigen transfectant). The cell expressing the target antigen may be a syngeneic cell or allogeneic cell. Preferably, the cell naturally expressing the target antigen is a syngeneic cell. Preferably, the target antigen transfectant is an allogeneic cell. The target antigen-expressing cell also may naturally or artificially express CD1d, or have no expression of CD1d. When the cell naturally or artificially expresses CD1d, the transfection of the cells with a nucleic acid (e.g., mRNA or vector) encoding CD1d can be used to enhance the expression of CD1d.

In a third embodiment, a cell co-expressing a target antigen and CD1d is prepared by transfecting one or two molecular species of a nucleic acid (e.g., mRNA or vector) encoding a target antigen and CD1d into a cell having no expressions of a target antigen and CD1d. The cell may be a syngeneic cell or allogeneic cell. Preferably, the cell is an allogeneic cell (in view of the large-scale production).

The cell expressing CD1 and target antigen of the present invention can be any suitable cell, such as an artificial adjuvant vector cell (aAVC). Preferably, the cell for the transfection in the present invention has at least one of the following properties: (1) a cell having an ability to express a high level of a protein encoded in the transfected nucleic acid (HEK293 cells and or HeLa cells have this property); and/or (2) a cell showing a high efficiency of transfection (no less than 50%) when a nucleic acid (e.g., mRNA or a vector) is transfected into the cell.

In one embodiment, the cell of the present invention is characterized in that it is a cell derived from the same individual to be immunized with the cell, that is an autologous cell to the subject of administration. In the present invention, such a cell is also called as “autologous cell” or “syngeneic cell”. In one embodiment, the cell of the present invention is characterized in that it is a cell derived from another individual of the same race to an individual to be immunized with the cell, that is a cell allogeneic to the subject of administration. In the present invention, such a cell is also called as “allogeneic cell” or “allo-cell”. An allo-cell may be preferred in view of the large-scale production. The cell co-expressing a target antigen and CD1d, which is provided by the present invention, is also referred to as the aAVC of the present invention.

CD1d ligand refers to a substance capable of activating NKT cells, presented on a CD1d-expressing antigen presenting cell (APC). Any CD1d ligand can be used in the present invention. Examples of “CD1d ligand” include α-GalCer (α-galactosylceramide), α-C-GalCer (α-C-galactosylceramide), iGB3 (isoglobotrihexosylceramide), GD3 (ganglioside 3), GSL-1 (α-linked glucuronic acid), GSL-1′SA (galacturonic acid), and α-GalCer derivatives described in references, but by no means exhaustive list of such references, including Morita et al., J. Med. Chem., 38:2176 (1995); Sakai et al., J. Med. Chem., 38:1836 (1995); Morita et al., Bioorg. Med. Chem. Lett., 5:699 (1995); Takakawa et al., Tetrahedron, 54:3150 (1998); Sakai et al., Org. Lett., 1:359 (1998); Figueroa-Perez et al., Carbohydr. Res., 328:95 (2000); Plettenburg et al., J. Org. Chem., 67:4559 (2002); Yang et al., Angew. Chem., 116:3906 (2004); Yang et al., Angew. Chem. Int. Ed., 43:3818 (2004); and Yu et al., Proc. Natl. Acad. Sci. USA, 102(9):3383-3388 (2005); U.S. Pat. No. 5,936,076 to Higa et al., and U.S. Pat. No. 6,531,453 to Taniguchi et al., U.S. Pat. No. 5,853,737 to Modlin et al., U.S. Pat. No. 7,488,491 to Tsuji et al., U.S. Patent Application Publication 2003-030611 to Jiang et al., U.S. Patent Application Publication 2004-0242499 to Uematsu et al., U.S. Patent Application Publication 2010-0062990 to Tashiro et al., U.S. Patent Application Publication 2011-0104188 to Tashiro et al., U.S. Patent Application Publication 2011-0224158 to Shiozaki, International Patent Application Publication WO 2003/105769 to Tsuji et al., International Patent Application Publication WO 2005/102049 to Tsuji et al., International Patent Application Publication WO 2007/137258 to Tsuji et al., International Patent Application Publication WO 2008/005824 to Teyton et al., International Patent Application Publication WO 2008/082156 to Kang et al., International Patent Application Publication WO 2008/128207 to Wong et al., International Patent Application Publication WO 2006/026389 to Porcelli, International Patent Application Publication WO 2009/060305 to Panza, International Patent Application Publication WO 2006/071848 to Wong et al., International Patent Application Publication WO 2007/050668 to Cerundolo et al., International Patent Application Publication WO 2007/074788 to Oku et al., International Patent Application Publication WO 2007/105115 to Galli, with preference given to α-GalCer and α-C-GalCer. The aAVC of the present invention presents CD1d ligand on its cell surface via CD1d, and can activate NKT cells.

Moreover, when a nucleic acid (e.g., mRNA and/or vector) encoding the target antigen and/or CD1d is transfected, the aAVC of the present invention can highly express a protein encoded therewith. Examples of such cell include a cell that affords EGFP positive cells in a proportion of preferably not less than 50%, more preferably not less than 60%, of the whole cells; when 5 μg of mRNA encoding EGFP protein is transfected into the cells (2×10⁵ cells) by using a TransMessenger transfection kit (Qiagen) or by the operation of the electroporation according to the protocol thereof and identified by FACS analysis 4 hr later. A cell in which transfection efficiency of target antigen mRNA and expression efficiency of its protein are high level may also be used even if transfection efficiency of EGFP mRNA and expression efficiency of EGFP protein are low level. When the expression efficiency of the protein encoded by mRNA to be transfected depends on the culture conditions and the like, optimal transfection conditions are determined by experiment, based on which the cell of the present invention can be produced.

The present invention also provides a cell from among predetermined cells (e.g., target antigen and CD1d co-expressing cells) that can be loaded with CD1d ligand. The present inventors have found for the first time that an aAVC (a cell transfected with mRNA encoding the target antigen and co-expressing a target antigen and CD1d) that is loaded (pulsed) with a CD1d ligand is highly useful for immunotherapy.

A cell that is “loaded” or “pulsed” (i.e., a “loaded cell” or a “pulsed cell”) is a cell that is cultured with CD1d ligand, wherein the CD1d ligand is bound to the CD1d of the cell. The culturing conditions can be any suitable conditions. For example, the cell can be cultured with CD1d ligand for 6 hours or more (e.g., 7 hours, 8 hours, 9 hours, 10 hours, 11 hours or 12 hours). Preferably, co-culture time is more than 8 hours. More preferably, co-culture time is more than 12 hours. From among the aAVCs of the present invention, the cell loaded with CD1d ligand is referred to as “loaded cell of the present invention” or “pulsed cell of the present invention” (which means a cell that may have an ability to activate immunity against the target antigen) as necessary. Furthermore, from among the aAVCs of the present invention, the cell which is not loaded with a CD1d ligand is referred to as “unloaded cell of the present invention” or “non-pulsed cell of the present invention” (which means a cell that can acquire an ability to activate immunity against the target antigen after being loaded with a CD1d ligand, but does not have an ability to activate immunity against the target antigen since it is not loaded with CD1d ligand) as necessary.

The aAVC of the present invention may be isolated and/or purified. Cell isolation and purification can be performed by a method known per se.

The aAVC of the present invention may also be derived from any animal species. Examples of such animal species include mammals such as human, monkey, chimpanzee, dog, cat, horse, bovine, swine, sheep, goat, mouse, rat, guinea pig, hamster, rabbit and the like, with preference given to a cell derived from human from the aspect of clinical application (syngeneic or allogeneic for the human subject).

Furthermore, the base cell of the aAVC of the present invention may be a cell type derived from any tissue. Examples of such tissue include stomach, small intestine (e.g., duodenum, jejunum, ileum, colon), large intestine, rectum, lung, pancreas, kidney, liver, thymus, spleen, thyroid gland, adrenal gland, prostate, ovary, uterus, bone marrow, skin, and peripheral blood. The aAVC of the present invention may also be a particular cell type in the above-mentioned tissues or a cell type in a tissue other than the above-mentioned tissues. Examples of such cell type include epithelial cell, endothelial cell, epidermal cell, interstitial cell, fibroblast, adipocyte, mammary cell, mesangial cell, pancreatic β cells, nerve cell, glial cell, immune cell (e.g., T cell, B cell, NK cell, NKT cell, macrophage, mast cell, neutrophil, basophil, eosinophils, monocyte), and precursor cells and stem cells of these cells.

Furthermore, the base cell of the aAVC of the present invention may be a cell obtained from an animal (e.g., primary cultured cell) or a cell line. The cell line may be an existing cell line or a newly prepared cell line (e.g., HEK293 or HeLa). The cell line can be prepared by a method known per se.

More importantly, the aAVC of the present invention may be a cell that expresses both a target antigen and CD1d.

A target antigen is an antigen expressed on an abnormal cell or pathogen, and is not particularly limited as long as intracorporeal disappearance of the abnormal cell or pathogen, or a decrease in the amount of the abnormal cell or pathogen is expected by an immune reaction against the target antigen. Examples of the target antigen include tumor/cancer antigen and pathogenic antigen. The cell of the present invention can express one or more target antigens to the same target.

The tumor antigen may be an antigen of a solid tumor including epithelial and nonepithelial tumors, or a tumor in a hematopoietic tissue. Those skilled in the art recognize that tumor cells have various antigens, and that tumor cells have a wide variety of properties. The tumor antigen for use in the present invention may be selected from those expressed in tumor cells derived or isolated from the subject to be immunized with the aAVC. In such a case, it is preferred that the selected tumor antigen is expressed at high level in the tumor cells compared with normal cells of the subject. A tumor antigen also may be selected from tumor antigens that have been identified in a tumor cell targeted for an immunization with an aAVC. While the tumor antigen (e.g., solid tumor antigen) is not particularly limited, for example, MART-1/Melan-A, Mage-1, Mage-2, Mage-3, Mage-4, Magea2, Magea3, Magea4, gp100, tyrosinase, tyrosinase-related protein 2 (trp2), CEA, PSA, CA-125, erb-2, Muc-1, Muc-2, TAG-72, AES, FBP, C-lectin, NY-ESO-1, galectin-4/NY-CO-27, Pec60, HER-2/erbB-2/neu, telomerase, G250, Hsp105, point mutated ras oncogene, point mutated p53 oncogene and carcinoembryonic antigen can be mentioned (e.g., JP-A-2005-139118, JP-A-2004-147649, JP-A-2002-112780, JP-A-2004-222726). While the antigen of tumor in a hematopoietic tissue (e.g., leukemia) is not particularly limited, for example, proteinase 3, WT-1, hTERT, PRAME, PML/RAR-a, DEK/CAN, cyclophilin B, TEL-MAL1, BCR-ABL, OFA-iLRP, Survivin, idiotype, Sperm protein 17, SPAN-Xb, CT-27 and MUC1 can be mentioned.

The pathogenic antigen may be a pathogenic virus antigen, a pathogenic microorganism antigen, or a pathogenic protozoan antigen. While a pathogenic virus antigen is not particularly limited, for example, antigens of viruses such as human immunodeficiency virus (HIV), hepatitis virus (e.g., type A, type B, type C, type D and type E hepatitis virus), influenza virus, simple herpes virus, West Nile fever virus, human papillomavirus, horse encephalitis virus, human T-cell leukemia virus (e.g., HTLV-I) and the like can be mentioned. Specifically, for example, GP-120, p17, GP-160 (HIV); NP, HA (influenza viruses); HBs Ag, HBV envelope protein, core protein, polymerase protein, NS3, NS5 (hepatitis viruses); HSVdD (simple herpes virus); EBNA1, 2, 3A, 3B and 3C, LMP1 and 2, BZLF1, BMLF1, BMRF1, BHRF1 (EB viruses); Tax (HTLV-I); SARS-CoV spike protein (SARS virus); CMV pp5, IE-1 (CMVs); E6, E7 proteins (HPVs) can be mentioned (e.g., JP-A-2004-222726). Examples of the pathogenic microorganism antigen include antigens expressed in pathogenic bacterium (e.g., chlamydiae, mycobacteria, Legionella) or pathogenic yeast (e.g., aspergillus, Candida). Examples of the pathogenic protozoan antigen include antigens expressed in malaria or schistosome.

CD1d is known to be a major histocompatibility complex (MHC)-like molecule that presents glycolipid rather than peptide. CD1d is also expressed in antigen presenting cell (e.g., dendritic cell), epithelial cells in tissues in the intestine, liver and the like, some tumor cells (e.g., solid tumor cell, leukemia cell) and virus-infected cells. CD1d is highly conserved among the mammals (e.g., human CD1d; NM_{—}001766, mouse CD1d; NM_{—}007639). It is known that human NKT cell can be activated by CD1d ligand presented by mouse CD1 homologues (Brossay et al., J. Exp. Med., 188: 1521-1528 (1998)). The CD1d of the present invention can be selected from the CD1d of any species of mammals. Preferably, the CD1d is human CD1d (including its highly-homologous derivative, variant or mutant equivalent to the function of human CD1d).

The aAVC of the present invention may be prepared from a normal cell or abnormal cell. The normal cell refers to a cell not in a pathogenic state. On the other hand, the abnormal cell refers to a cell in a pathogenic state. Preferably, an abnormal cell is derived or isolated from a subject to be immunized with the aAVC. Examples of the abnormal cell include a tumor cell and virus-infected cell. The tumor cell may be one corresponding to the aforementioned cell type. Examples of the virus-infected cell include the aforementioned cell type infected with a pathogenic virus. While the pathogenic virus is not particularly limited, for example, human immunodeficiency virus (HIV), hepatitis virus (e.g., type A, type B, type C, type D and type E hepatitis virus), influenza virus, simple herpes virus, West Nile fever virus, human papillomavirus, equine encephalitis virus and human T-cell leukemia virus (e.g., HTLV-I) can be mentioned.

The aAVC of the present invention may also be prepared from a non-transfectant or transfectant. When transfection is used herein, it refers to an artificial transgenic operation, and the transfectant means a cell produced by such artificial operation. Therefore, a cell produced by a non-artificial operation is treated as one not falling under a transfectant herein. To be more precise, when the cell of the present invention is a transfectant, the aAVC of the present invention can be produced by using a CD1d-expressing cell as a host cell and by transfecting the cell with a nucleic acid (e.g., mRNA or vector) encoding the target antigen. The host cell for transfection may be any cell expressing CD1d, and can be, for example, a cell naturally expressing CD1d or a cell prepared to express CD1d by an artificial operation. When the cell of the present invention is a target antigen transfectant, the aAVC of the present invention can be produced by using a target antigen-expressing cell as a host cell and by transfecting the cell with a nucleic acid (e.g., mRNA or vector) encoding CD1d. The host cell for transfection may be any cell expressing a target antigen, and can be, for example, a cell naturally expressing a target antigen or a cell prepared to express target antigen by an artificial operation. The transfectant in the present invention means both stable transfectant and non-stable transfectant (transient transfectant). The terms “transfect” and “introduce” are used interchangeability herein.

The term “nucleic acid” as used herein includes mRNA encoding a target antigen and/or CD1d, and a vector that encodes (expresses) the target antigen and/or CD1d. The nucleic acid including mRNA and vector may be used for the expression of, or the enhancement of, the expression of the target antigen and/or CD1d in a cell (e.g., the base cell of an aAVC). When mRNA is used for the preparation of an aAVC, the inventive cells, compositions, cells, and methods are highly safe and suppress, as much as possible, the possibility of side effects generally feared in gene therapy. In addition, the use of mRNA may be preferred in some countries and regions in view of the avoidance of gene therapy.

The loaded cell of the present invention is useful as a pharmaceutical agent, an immune activator and the like. The unloaded cell of the present invention is useful, for example, for the preparation of the loaded cell of the present invention.

(2. Preparation and Identification Methods)

The present invention provides a method for preparing the aAVC of the present invention.

In one embodiment, the preparation method of the present invention can be a method for preparing the unloaded cell of the present invention. The preparation method of the unloaded cell of the present invention may include treating a cell such that the target antigen and CD1d will be co-expressed in a base cell of an aAVC, or expression of the target antigen and/or CD1d will be enhanced in the cell co-expressing a target antigen and CD1d in a base cell of an aAVC. The base cell of an aAVC (object cell) is a cell that is allogeneic to a target in need of immunity induction prior to transfection of the nucleic acid encoding the target antigen and/or CD1d.

For example, when the preparation method of the present invention includes treatment of an object cell such that the target antigen and CD1d will be co-expressed in the cell, the object cell may be a cell that does not express both the target antigen and CD1d, a target antigen-expressing cell, or a CD1d-expressing cell.

In addition, when the preparation method of the present invention includes treatment of a cell such that the expression of the target antigen and/or CD1d will be enhanced in the cell co-expressing a target antigen and CD1d, the expression of the target antigen and/or CD1d can be enhanced to the degree that the treatment effect of the immunotherapy using the cell of the present invention is sufficiently increased.

The treatment in the preparation method of the present invention may be transfection or an operation to transfect a nucleic acid (e.g., mRNA or vector) of a target antigen and/or CD1d. To be more precise, the preparation method of the present invention can include (a) transfecting a nucleic acid (e.g., mRNA or vector) encoding a target antigen or CD1d into a cell expressing the target antigen or CD1d, (b) transfecting 1 or 2 molecular species of a nucleic acid (e.g., mRNA or vector) encoding a target antigen and CD1d into a cell. The transfection of the cell and a nucleic acid (e.g., mRNA or vector) transfection can be performed by a method known per se such as a lipofection method, a calcium phosphate precipitation method, an electroporation method and the like. Direct transfection of the target antigen mRNA and/or CD1d mRNA is preferred.

In the above-mentioned embodiment (b), the “1 molecular species of nucleic acid encoding a target antigen and CD1d” means both the target antigen and CD1d are encoded by one nucleic acid, and the “two molecular species of nucleic acid encoding a target antigen and CD1d” means each of the target antigen and CD1d is encoded by a separate nucleic acid.

Here, the target antigen to be expressed in an object cell may be one or more kinds. That is, a nucleic acid (e.g., mRNA or vector) of the target antigen to be used for the preparation method of the aAVC may be a nucleic acid (e.g., mRNA or vector) derived from one kind of antigen or a mixture of nucleic acids (e.g., mRNAs or vectors) derived from plural kinds of antigens (there are plural kinds of mRNA encoding the antigen).

In the aforementioned preparation method, the base cell (i.e., the cell prior to transfection) of the aAVC is selected from: (i) a CD1d-expressing cell, (ii) a target antigen-expressing cell, or (iii) a cell having no expressions of a target antigen and CD1d. The CD1d-expressing cell may be (i-i) a cell naturally expressing CD1d or (i-ii) a cell previously transfected with a nucleic acid (e.g., mRNA or vector) encoding CD1d (CD1d transfectant). The target antigen-expressing cell may be (i-i) a cell naturally expressing target antigen or (i-ii) a cell previously transfected with a nucleic acid (e.g., mRNA or vector) encoding target antigen (target antigen transfectant).

In another embodiment, the preparation method of the present invention may be a preparation method of the loaded cell of the present invention. The preparation method of the loaded cell of the present invention may include treating (contacting, e.g., by loading or pulsing) a cell co-expressing a target antigen and CD1d, for example the unloaded cell of the present invention, with a CD1d ligand in a culture medium. The culture condition can be culturing with CD1d ligand for 6 hours or more (e.g., 7 hours, 8 hours, 9 hours, 10 hours, 11 hours or 12 hours). Preferably, co-culture time is more than 8 hours. More preferably, co-culture time is more than 12 hours. By such a treatment, the CD1d ligand is presented on the cell co-expressing a target antigen and CD1d, and the co-expressing cell can acquire an ability to activate immunity against the target antigen.

The culture medium can be prepared using, as a basal medium, a medium used for culturing animal cells. Examples of the basal medium include MEM medium, DMEM medium, αMEM medium, Ham's medium, RPMI1640 medium, Fischer's medium, and a mixed medium thereof. The culture medium can contain, for example, serum (e.g., FCS), serum replacement (e.g., knockout Serum Replacement (KSR)), fatty acid or lipid, amino acid, vitamin, growth factor, cytokine, antioxidant, 2-mercaptoethanol, pyruvic acid, buffering agent, inorganic salts and the like. Other culture conditions such as culture temperature, CO₂ concentration and the like can be set as appropriate. While the culture temperature is not particularly limited, for example, it is about 30-40° C., preferably about 37° C. The CO₂ concentration is, for example, about 1-10%, preferably about 5%. Other conditions such as the number of cells to be cultured, concentrations of various factors and the like can be appropriately set by methods known per se.

When the treated cell (e.g., transfectant) is used as a cell co-expressing a target antigen and CD1d, the preparation method of the present invention may further include treating a cell such that the target antigen and CD1d will be co-expressed in the cell (object cell) or the expression of the target antigen and/or CD1d will be enhanced in the cell co-expressing a target antigen and CD1d, and obtaining the cell co-expressing a target antigen and CD1d. The methodology is the same as for the aforementioned preparation method of the present invention.

When an untreated cell (e.g., non-transfectant) is used as a target antigen and CD1d co-expressing cell, the preparation method of the present invention may further include recovering the target antigen and/or CD1d co-expressing cell from a biological sample obtained from an individual to give the target antigen and CD1d co-expressing cell. Examples of the biological sample obtained from an individual include biological samples such as tumor mass, peripheral blood, liver, lymph node, spleen and the like. Examples of the cell obtained from an individual include abnormal cells such as tumor cells (e.g., leukemia cell), virus-infected cell and the like (the abnormal cell may be of the same type as the cell of the present invention). The individual may be of the same type as the aforementioned animal species. The individual may also be the same as or different from the individual to be administered with the cell of the present invention.

The inventive methods may include measurement of expression of CD1d, expression of a target antigen, or expressions of target antigen and CD1d in the object cell. Examples of the object cell include treated cell and untreated cell, and existing cell and the inventive aAVC. Examples of the object cell and/or a cell obtained from an individual include abnormal cells such as tumor cell, virus-infected cell and the like.

The expression of the target antigen and CD1d can be measured by a method known per se and using, for example, plural primers (e.g., primer pair(s)) that can amplify RNA of target antigen and/or CD1d, nucleic acid probe that can detect RNA of target antigen and/or CD1d, or an antibody to target antigen and/or CD1d.

(3. Construct for In Vitro Transcription and mRNA)

The present invention provides a construct for in vitro transcription to produce a nucleic acid (e.g., mRNA) encoding a target antigen. Examples of the construct include a template construct transcribing only a target antigen mRNA, and a template construct transcribing a target antigen mRNA and mRNA of other useful factor. When plural kinds of target antigen mRNAs are used, respective mRNAs may be present in the same construct or separately present in different constructs. Examples of the construct for transcription in vitro to produce an mRNA encoding the target antigen include a template construct derived from a target antigen expression vector, and a template construct derived from a vector for co-expression of a target antigen and CD1d.

Similarly, a construct for in vitro transcription of a nucleic acid (e.g., mRNA) encoding CD1d is provided. Examples of the construct include a template construct for expression of only CD1d mRNA, and a template construct for expression of CD1d mRNA and mRNA of the other useful factor. Examples of the construct for in vitro transcription to produce an mRNA encoding CD1d include a template construct derived from a CD1d-expression vector, and a template construct derived from a vector for co-expression a target antigen and CD1d.

In the same manner, a construct for in vitro transcription of 1 or 2 molecular species of a nucleic acid (e.g., mRNA) encoding a target antigen and CD1d is provided. Examples of the construct include a template construct for expression of both a target antigen mRNA and CD1d mRNA, and a combination of a template construct for expression of at least a target antigen mRNA and a template construct for expression of at least CD1d mRNA. Examples of the construct for in vitro transcription to produce of 1 or 2 molecular species mRNA encoding a target antigen and CD1d include a template construct derived from a vector for co-expression of a target antigen and CD1d, and a combination of a template construct derived from a target antigen-expression vector and a template construct derived from a CD1d-expression vector. When plural kinds of target antigen mRNAs are used, respective mRNAs may be present in the same construct or separately present in different constructs.

The present invention also provides a construct for co-expression of such nucleic acids (e.g., mRNAs).

The co-expression construct of the present invention may contain a first polynucleotide encoding a target antigen and a second polynucleotide encoding CD1d. When plural kinds of target antigens are the targets, respective target antigens may be contained in the same polynucleotide or separately contained in different polynucleotides. The co-expression construct of the present invention may also contain a promoter operably linked to the above-mentioned first and the second polynucleotides. The operable linkage of the promoter means that the promoter is bound to the polynucleotide in such a manner as to permit expression of a factor encoded by the polynucleotide under regulation of the promoter.

To be more precise, the co-expression construct of the present invention may be a polycistronic mRNA expression construct. The polycistronic mRNA expression construct may contain a conjugate of a first polynucleotide (encoding one or more target antigens) and second polynucleotide, which enables expression of polycistronic mRNA of one or more target antigens and CD1d, and a promoter operably linked to the conjugate.

The co-expression construct of the present invention may also be a non-polycistronic mRNA expression vector. The non-polycistronic mRNA expression construct may contain a first polynucleotide and a first promoter operably linked to the polynucleotide, as well as a second polynucleotide and a second promoter operably linked to the polynucleotide. When plural kinds of target antigens are the targets, respective target antigens may be contained in the same polynucleotide or separately contained in different polynucleotides.

The promoter to be used for the construct for in vitro transcription is not particularly limited as long as it is operable in vitro and, for example, T7 promoter, SP6 promoter, T3 promoter and the like can be mentioned.

The construct for in vitro transcription preferably contains a transcription termination signal, i.e., terminator region, at the downstream of oligo(poly)nucleotide encoding a nucleic acid molecule. From the aspects of stability of synthesized mRNA and the like, moreover, it preferably contains a polyA sequence. Examples of the above-mentioned terminator sequence include SP6 terminator, T7 terminator, T3 terminator and the like. To amplify the co-expression construct itself, it may further contain a selection marker gene (gene imparting resistance to pharmaceutical agents (e.g., tetracycline, ampicillin, kanamycin, hygromycin, phosphinothricin), gene complementing auxotrophic mutation etc.) for selecting a transfected cell, which functions as a vector. For amplification, PCR may also be used.

When the construct for transcription in vitro is derived from a vector, the backbone thereof may be derived from, for example, a plasmid or a virus vector (e.g., vector derived from virus such as adenovirus, retrovirus, adeno-associated virus, herpesvirus, vaccinia virus, poxvirus, polio virus, sindbis virus, Sendai virus, lentivirus and the like).

The mRNA encoding the target antigen and the mRNA encoding CD1d, or the mRNA encoding target antigen and CD1d (hereinafter to be also referred to simply as the mRNA of the present invention) to be used in the present invention are prepared by a known method using the above-mentioned construct of the present invention and a commercially available in vitro transcription kit and the like. The template construct to be contained in the transcription reaction mixture may be circular or linear. When it is a circular DNA, it may be linearized by digestion with a restriction enzyme that recognizes the restriction enzyme site at an appropriate position. The template constructs to be used in the present invention are not particularly limited in the number of bases, and they may not have the same number of bases as long as the object protein can be synthesized. As long as it is a sequence homologous to the degree permitting synthesis of the object protein, moreover, each template construct may contain plural bases which have been deleted, substituted, inserted or added. From the aspects of stability, it desirably has a 5′ cap structure.

The inventive nucleic acid (mRNA) is useful for the activation of immunocytes and preparation of the inventive aAVC.

In the present invention, the aforementioned target antigen expression vector, aforementioned CD1d-expression vector, and aforementioned vector for co-expression of a target antigen and CD1d can be utilized to prepare a cell co-expressing a target antigen and CD1d as a nucleic acid to express or to enhance the expression of a target antigen and/or CD1d.

(4. Agent and Composition)

The present invention provides an agent (or composition) containing the aAVC of the present invention, particularly the loaded cell of the present invention loaded with a CD1d ligand. The agent or composition can be used in a method of inducing immunity. Therefore, the agent and composition containing the aAVC of the present invention is an immunoinducer.

Accordingly, the present invention provides a method of inducing immunity comprising administering an effective amount of the aAVC (or agent or composition thereof) to a subject in need thereof, wherein the cell is syngeneic or allogeneic to the subject.

The subject can be any suitable subject. For example, the subject can be animal species including mammals such as human, monkey, chimpanzee, dog, cat, horse, bovine, swine, sheep, goat, mouse, rat, guinea pig, hamster, rabbit and the like. In one embodiment, the subject is a non-human subject.

The subject to which the aAVC (or agent or composition thereof) is administered may be the same as the animal species from which the aAVC of the present invention is derived. Thus, the agent of the present invention can achieve alloimmunization in the immunization with the aAVC of the present invention when the allogeneic cell is used.

The agent of the present invention may contain, in addition to the loaded cell of the present invention, any carrier, for example, pharmaceutically acceptable carriers and/or adjuvant. Examples of the pharmaceutically acceptable carrier include, but are not limited to, diluents such as water, saline and the like. While the adjuvant is not particularly limited as long as it can enhance the antigenicity of the target antigen, for example, BCG, trehalose dimycolate (TDM), Merck65, AS-2, aluminum phosphate, aluminum hydroxide, keyhole limpet hemocyanin, dinitrophenol, dextran and TLR ligand (e.g., lipopolysaccharide (LPS), CpG) can be mentioned.

The agent of the present invention is useful, for example, as a pharmaceutical agent or reagent. To be more precise, the agent of the present invention is useful for the prophylaxis or treatment of neoplastic diseases or infections, or immunotherapy (e.g., activation of immunocytes such as NK/NKT cells, T cells and the like). Examples of the neoplastic diseases possibly prevented or treated by the agent of the present invention include tumors in the aforementioned tissues and cell types, such as solid tumor (e.g., epithelial tumor, non-epithelial tumor), and tumors in hematopoietic tissues. To be more precise, examples of the solid tumor possibly prevented or treated by the agent of the present invention include digestive organ cancer (e.g., gastric cancer, colon cancer, colorectal cancer, rectal cancer), lung cancer (e.g., small cell cancer, non-small cell cancer), pancreatic cancer, kidney cancer, liver cancer, thymus, spleen, thyroid cancer, adrenal gland cancer, prostate cancer, urinary bladder cancer, ovarian cancer, uterus cancer (e.g., endometrial carcinoma, cancer of the uterine cervix), bone cancer, skin cancer, sarcoma (e.g., Kaposi's sarcoma), melanoma, blastoma (e.g., neuroblastoma), adenocarcinoma, planocellular carcinoma, non-planocellular carcinoma, brain tumor, as well as recurrence and metastasis of these solid tumors. Examples of the tumor in the hematopoietic tissue possibly prevented or treated by the agent of the present invention include leukemia (e.g., acute myeloid leukemia (AML), chronic myelocytic leukemia (CML), acute lymphocytic leukemia (ALL), chronic lymphocytic leukemia (CLL), adult T cell leukemia (ATL), myelodysplastic syndrome (MDS)), lymphoma (e.g., T lymphoma, B lymphoma, Hodgkin's lymphoma), myeloma (multiple myeloma), as well as recurrence of these tumors. Examples of the infections possibly treated by the agent of the present invention include infections caused by the aforementioned pathogens.

The present inventors have also found at this time that the loaded cell of the present invention can have an ability to simultaneously induce activation of NK/NKT cells and T-cell immune response. It is known that MHC class I-non-expressing cell is a good target for NK/NKT cells, and MHC class I-expressing cell is a good target for T cells. Accordingly, the agent of the present invention is advantageous in that an effect on various target antigen-expressing cells can be expected.

While the dose of the agent of the present invention varies depending on the kind of the loaded cell of the present invention, expression levels of the target antigen and CD1d in the loaded cell of the present invention, administration mode, severity of disease, animal species of the subject of administration, and acceptability, body weight, age and the like of the subject of administration and it cannot be generalized, cells in a number that affords a desired immunoactivity can be appropriately administered. The agent (or composition) of the present invention can also be used as a vaccine, and can also be used in a method of inducing immunity against a target antigen. When targeting plural kinds of target antigens (for example, when plural target antigen-derived nucleic acids (e.g., mRNAs or vectors) are transfected into an object cell etc.), an aAVC presenting polyvalent antigen can be obtained, and such aAVC can be used as a polyvalent vaccine, and in a method for inducing immunity against plural target antigens.

Examples of the target antigen nucleic acid (e.g., mRNA or vector) used for preparation of the agent (including vaccine) of the present invention include, but are not limited to, nucleic acids (e.g., mRNAs or vectors) encoding melanocyte differentiation antigen and tyrosinase-relating protein 2 (trp2). The aAVC of the present invention is prepared using a nucleic acid (e.g., mRNA or vector) encoding target antigen, and the loaded cell of the present invention is prepared by loading (pulsing) a CD1d ligand, and the obtained cell is used for immunization of an individual, whereby the growth of a cell characteristically containing the target antigen in the body can be specifically and markedly suppressed.

(5. Kit)

The above-mentioned various substances and/or cells can be formed as a kit as necessary.

To be more precise, the kit of the present invention is largely divided into a kit containing, as essential constituent components, a component for expressing a target antigen and a component for expressing CD1d (kit I), a kit further containing a CD1d ligand as an essential constituent component (kit II), and a kit further containing an expression measurement means as an essential constituent component (kit III).

The kit I of the present invention may contain, for example, any of (1) to (8) below:

(1) a combination of (1-1) a CD1d-expressing cell, and (1-2) a construct for in vitro transcription of a nucleic acid (e.g., mRNA or vector) encoding the target antigen;

(2) a combination of (2-1) a CD1d-expressing cell, and (2-2) a nucleic acid (e.g., mRNA or vector) encoding the target antigen;

(3) a combination of (3-1) a construct for in vitro transcription of a nucleic acid (e.g., mRNA or vector) encoding CD1d, (3-2) a construct for in vitro transcription of a nucleic acid (e.g., mRNA or vector) encoding the target antigen, and (3-3) an object cell;

(4) a combination of (4-1) a construct for in vitro transcription of a nucleic acid (e.g., mRNA or vector) encoding CD1d and a nucleic acid (e.g., mRNA or vector) encoding the target antigen, and (4-2) an object cell;

(5) a combination of (5-1) a construct for in vitro transcription of a nucleic acid (e.g., mRNA or vector) encoding CD1d, (5-2) a nucleic acid (e.g., mRNA or vector) encoding the target antigen, and (5-3) an object cell;

(6) a combination of (6-1) a nucleic acid (e.g., mRNA or vector) encoding CD1d and the target antigen, (6-2) a construct for in vitro transcription of a nucleic acid (e.g., mRNA or vector) encoding the target antigen, and (6-3) an object cell;

(7) a combination of (7-1) a nucleic acid (e.g., mRNA or vector) encoding CD1d and the target antigen, (7-2) a nucleic acid (e.g., mRNA or vector) encoding the target antigen, and (7-3) an object cell; and

(8) a combination of (8-1) a nucleic acid (e.g., mRNA or vector) encoding CD1d and the target antigen, and (8-2) an object cell.

The kit II of the present invention contains, in addition to the respective constitution components of the above-mentioned kit I, a CD1d ligand as an essential constitution component. The CD1d ligand to be contained in kit II is similar to those mentioned above. Preferred are α-GalCer and α-C-GalCer.

The kit III of the present invention may contain a means capable of measuring the expression of a target antigen, and a means capable of measuring the expression of CD1d. Examples of the means capable of measuring the expression of a target antigen include antibody.

The promoter used for in vitro nucleic acid (e.g., mRNA) transcription, a construct to be the backbone and other factors for the kit of the present invention may be the same as those used for the construct of the present invention.

The CD1d-expressing cell to be contained in the kit of the present invention may be a cell that expresses CD1d and does not express a target antigen. The object cell to be contained in the kit of the present invention may be a cell that does not express CD1d and a target antigen, or a cell that expresses CD1d only at a low expression level and does not express a target antigen. The cell to be used in the present invention is characteristically a cell derived from another individual allogeneic to an individual to be immunized with the cell, that is, a cell allogeneic to subject of administration (allo-cell).

The kit of the present invention may further contain the aforementioned adjuvant.

The kit of the present invention may also contain a reagent that can confirm activation of an immunocytes. Examples of the reagent that can confirm activation of immunocytes include a reagent for the measurement of the number of one or more immunocytes selected from the group consisting of NK cell, NKT cell and T cell, and a reagent for the measurement of a substance specific to the activated immunocyte.

The reagent for the measurement of the number of immunocytes may contain, for example, specific antibodies against cell surface markers (e.g., Vα24, Vβ11) of NK cell, NKT cell and T cell, or nucleic acid probes capable of detecting transcription products encoding the markers or plural primers (e.g., primer pair(s)) capable of amplifying them.

The reagent for the measurement of a substance specific to the activated immunocyte may contain, for example, antibodies against substances (e.g., IFN-γ, perforin, granzyme B) specific to activated NK cell, NKT cell or T cell, or nucleic acid probes capable of detecting transcription products encoding the substances or plural primers (e.g., primer pair(s)) capable of amplifying them.

The kit of the present invention is, like the aforementioned agent of the present invention, useful, for example, as a kit for a pharmaceutical agent, or a kit for activation of immunocyte, or a kit for the preparation or identification of the cell of the present invention.

In one embodiment, the a CD1d ligand known (CD1d ligand) is loaded into an allogeneic fibroblast, rather than its autologous dendritic cell or tumor cell, transfected with a nucleic acid (e.g., mRNA or vector) encoding the target antigen, and the cell thereof is administered to activate NKT cell and NK cell in vivo, whereby a sufficient treatment effect can be obtained. Furthermore, according to the present invention, an immunotherapy against a wide range of therapeutic targets can be established by identifying mRNA in a target tumor cell or pathogen-infected cell once a small amount of these cells can be obtained. According to the present invention, moreover, a cell capable of activating immunity against the target antigen can be prepared by selecting an aAVC capable of highly efficient expression of a protein from a transfected nucleic acid (e.g., mRNA) and by transfecting the cell with a nucleic acid (e.g., mRNA) alone. When mRNA is used instead of a vector to deliver the target antigen, the immunotherapy is not dealt with as a gene therapy by not using a virus vector, therefore, this immunotherapy is not associated with a fear of side effects caused by modification of the cell genome

The following examples further illustrate the present invention but, of course, should not be construed as in any way limiting its scope.

### Example 1

This example describes the materials and methods for Examples 2-8.

Preparation of Tumor Cells Pulsed with α-GalCer

As the tumor cell line pulsed with α-GalCer, mouse-derived melanoma cell line B16 and mouse-derived T lymphoma cell line EL4 were used. α-GalCer was added to B16 (2×10⁴ cells/ml) or EL4 (1×10⁵ cells/ml) to a concentration of 500 ng/ml, and the cells were cultured at 5% CO₂, 37° C. As the culture medium, 10% FCS-containing RPMI (10 ml) was used. After culturing for 2 days, B16 or EL4 pulsed with α-GalCer was washed 4 times with PBS, and then collected.

Preparation of Dendritic Cells Pulsed with α-GalCer

Bone marrow cells were collected from the femur and shin bone of wild-type mice (C57BL/6, 6- to 8-week-old, female), and CD4, CD8, B220 or I-Ab positive cells were removed using antibodies and complements. The obtained cells were adjusted to 1×10⁶ cells/ml with GM-CSF (10 ng/ml) and 5% FCS-containing RPMI, and cultured in a 24 well plate. The medium was changed every two days, and α-GalCer was added to 100 ng/ml on day 6. On day 7, LPS (100 ng/ml) was further added, and the dendritic cells were matured. The cells were collected on day 8, and washed with PBS.

Preparation of Mononuclear Cells

The mononuclear cells were prepared from the spleen and liver. Spleen was removed from wild-type mice (C57BL/6, 6- to 8-week-old, female), filtered with a cell strainer, red blood cells were hemolyzed with ACK lysing buffer, and the mononuclear cells were washed to give spleen-derived mononuclear cells. In addition, the liver was removed from wild-type mice or Jα281 gene deficient mice (Vα14⁺NKT cell deficient mouse: e.g., see Fujii et al., The Journal of Experimental Medicine, 198: 267-279 (2003), and Fujii et al., The Journal of Experimental Medicine, 199: 1607-18 (2004)), filtered with a stainless mesh, and a mononuclear cell layer was separated using a percoll by the density gradient centrifugation method to give liver-derived mononuclear cells.

Production of Lung Metastatic Animal Model and Evaluation Thereof.

B16 melanoma cells were adjusted to 5×10⁵/200 μl with PBS, and intravenously administered to mice (C57BL/6, 6- to 8-week-old, female) from the tail vein. After 2 weeks, the lung was removed from the mice and the number of metastatic B16 cells in the lung was measured.

### Example 2

The example demonstrates in vitro activation of NKT/NK cells by tumor cells pulsed with CD1d ligand.

B16 or EL4 cells pulsed with α-GalCer or dendritic cells (control) pulsed with α-GalCer were co-cultured with mononuclear cells (fraction containing NKT/NK cells) derived from the liver of a wild-type mouse or Ja281 gene deficient mouse (Va14⁺NKT cell deficient mouse), and the level of IFN-γ in the culture supernatant was measured by ELISA and using an anti-IFN-γ antibody. Since IFN-γ is a substance specifically produced by activated NKT/NK cells, the level of IFN-γ in the culture supernatant is an index of activated NKT/NK cells.

In addition, an experiment similar to the above was performed using B16 and EL4 cells into which a CD1d expression retrovirus vector (produced by using mouse CD1d gene (GenBank Accession No.: NM-007639) had been introduced).

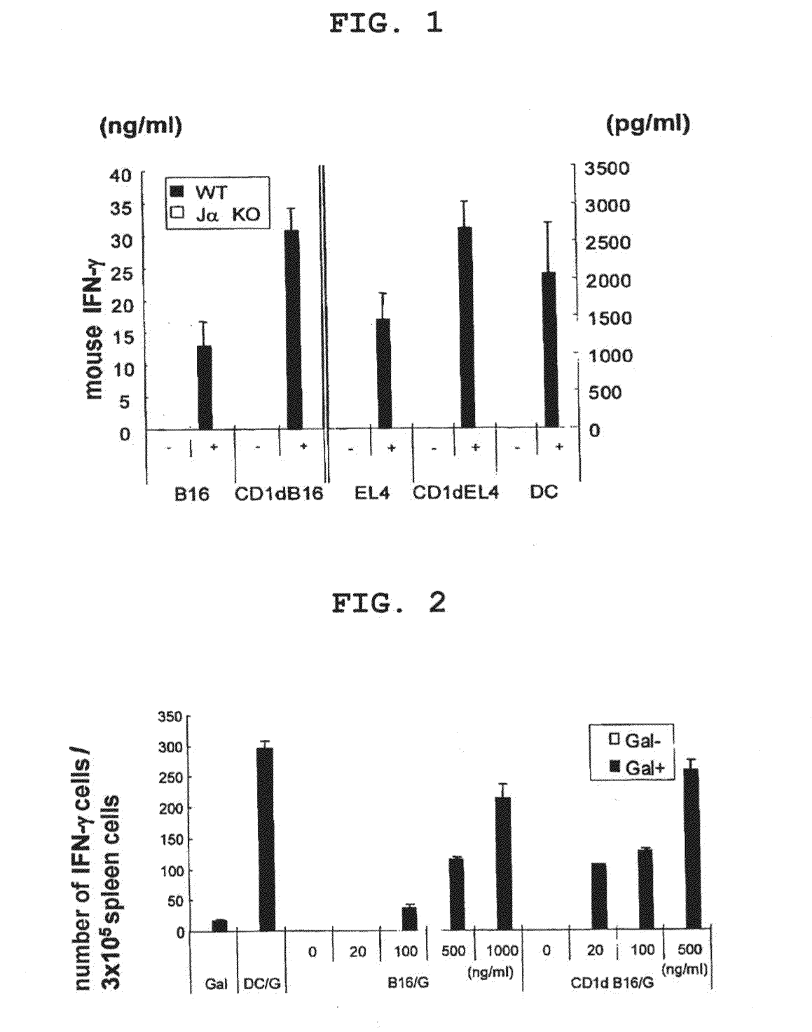

As a result, both B16 and EL4 cells pulsed with α-GalCer activated NKT/NK cells (see FIG. 1). In addition, B16 and EL4 cells pulsed with α-GalCer and containing a CD1d expression retrovirus vector activated NKT/NK cells more than B16 and EL4 cells pulsed with α-GalCer and free of the vector (see FIG. 1).

From the foregoing, it is clear that tumor cells pulsed with a CD1d ligand activate NKT/NK cells in vitro in a CD1d expression level-dependent manner.

### Example 3

The example demonstrates in vivo activation of NKT cells by tumor cells pulsed with CD1d ligand.

B16 cells (5×10⁵ cells) or dendritic cells (control, 1×10⁶ cells) pulsed with α-GalCer were intravenously administered to mice (C57BL/6, 6- to 8-week-old, female) in the tail vein. Two days after the administration, the spleen was isolated from the mice and filtered with a cell strainer, red blood cells were hemolyzed with ACK lysing buffer, and the splenocytes were adjusted with 5% FCS-containing RPMI. 3×10⁵ cells/well were cultured for 16 hr in the presence of α-GalCer in the same manner as in Example 2 and the level of IFN-γ in the culture supernatant was measured by ELISPOT and using an anti-IFN-γ antibody. In addition, an experiment similar to the above was performed using B16 cells into which a CD1d expression retrovirus vector had been introduced.

As a result, the dendritic cells pulsed with α-GalCer induced α-GalCer reactive IFN-γ producing NKT cells (see FIG. 1). When B16 cells were used, the efficiency decreased but α-GalCer reactive IFN-γ producing NKT cells could be induced in the same manner, where the induction capability was dependent on the concentration of α-GalCer used for pulsing and the induction capability was more efficient in B16 cells that strongly express CD1d (see FIG. 2).

From the foregoing, it is clear that tumor cells pulsed with a CD1d ligand activate NKT cells in vivo in an α-GalCer concentration-dependent manner.

### Example 4

This example demonstrates that antitumor effect on a lung metastatic animal model.

The antitumor effect of IFN-γ production by tumor cells pulsed with a CD1d ligand was examined. B16 cells, CD1d expression-enhanced B16 cells, B16 cells pulsed with α-GalCer, or CD1d expression-enhanced B16 cells pulsed with α-GalCer (5×10⁵ cells) were intravenously administered to mice. Then, after 14 days from the administration, the lungs were removed from the mice, and the antitumor effect was evaluated.

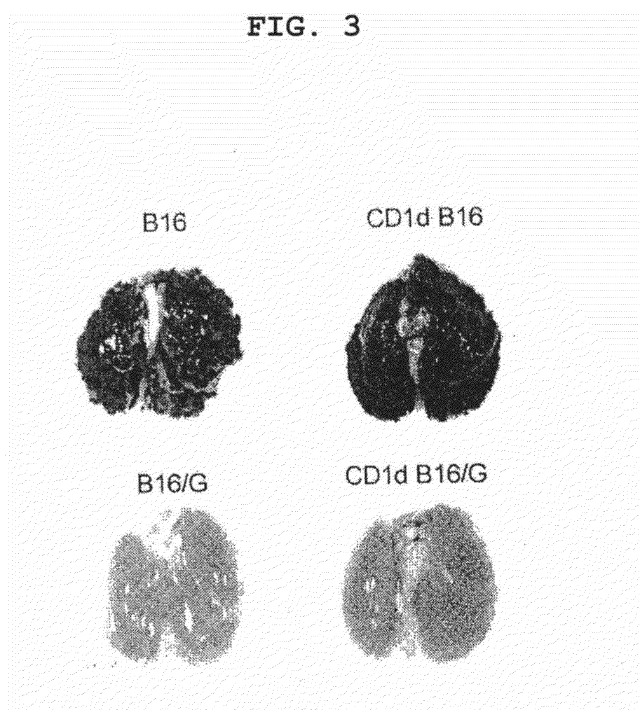

As a result, the metastatic model mice administered B16 cells or CD1d expression-enhanced B16 cells showed a tumor increase, but the tumor apparently disappeared in the model mice administered with B16 cells pulsed with α-GalCer or CD1d expression-enhanced B16 cells pulsed with α-GalCer (see FIG. 3).

From the foregoing, it is clear that tumor cells pulsed with a CD1d ligand show an antitumor effect by an enhanced spontaneous immune response.

### Example 5

This example demonstrates the induction of cytotoxic T lymphocyte to a tumor antigen.

B16 cells (5×10⁵ cells) were subcutaneously administered to a wild-type mouse administered CD1d expression-enhanced B16 cells pulsed with α-GalCer, whose tumor was confirmed to have disappeared in Example 4. In addition, B16 cells (1×10⁵ cells) were subcutaneously administered to a CD8 deficient mouse (purchased from Jackson Laboratory) administered with CD1d expression enhanced B16 cells pulsed with α-GalCer. The tumor resistance of these mice was compared.

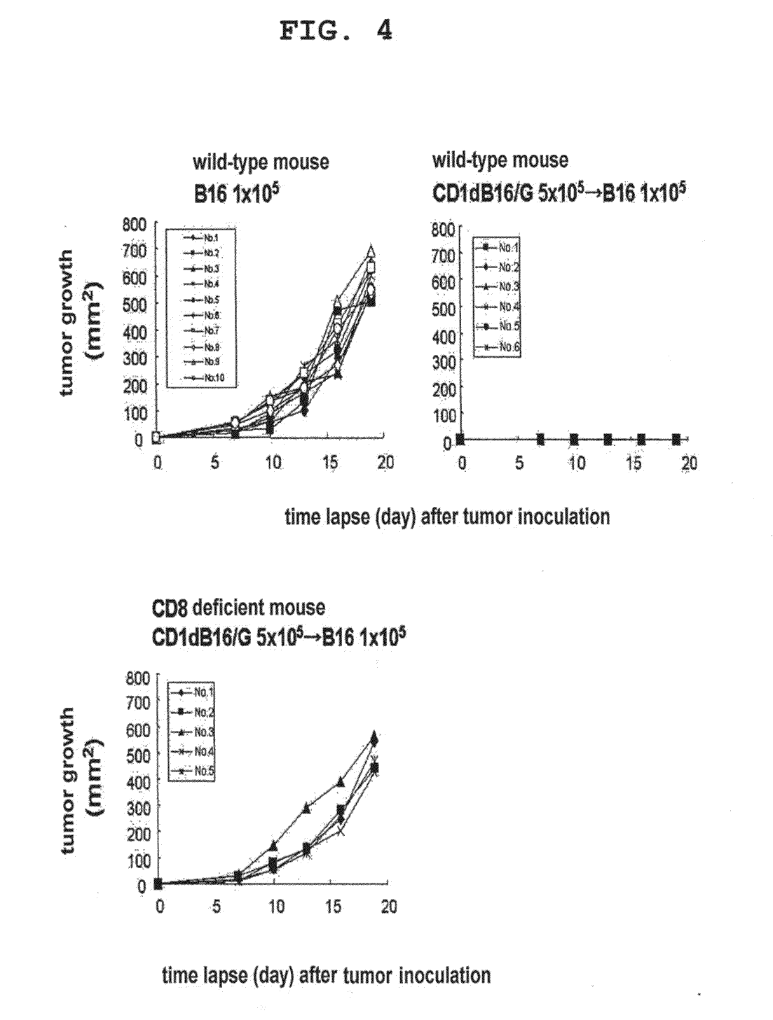

As a result, the wild-type mouse challenged with B16 cells showed resistance to the subcutaneously administered tumor. However, the tumor could not be eliminated in the CD8 deficient mouse (see FIG. 4). This shows that administration of a tumor cell pulsed with a CD1d ligand, such as α-GalCer, leads to the function of CD8⁺ cytotoxic T lymphocytes (CTL) as an effecter cell, whereby an antitumor effect is provided. Also, when administered to mice subcutaneously one year after the vaccine, parental B16 cells inoculated s.c. were rejected, suggesting the existence of memory T cells.

From the foregoing, it is clear that the inventive method can produce CTL to a tumor antigen expressed in the tumor. Furthermore, the inventive method can induce the immunological memory in the subject to be administered.

### Example 6

This example demonstrates the measurement of CD1d expression levels in tumor cell lines.

The CD1d expression levels were measured by real time RT-PCR and flow cytometry in mouse-derived melanoma cell line B16, mouse-derived T lymphoma cell line EL4, mouse-derived plasma cell (B cell) line J558, mouse-derived monocytic leukemia cell line WEHI-3B, and these cell lines harboring a CD1d gene introduced by a retrovirus.

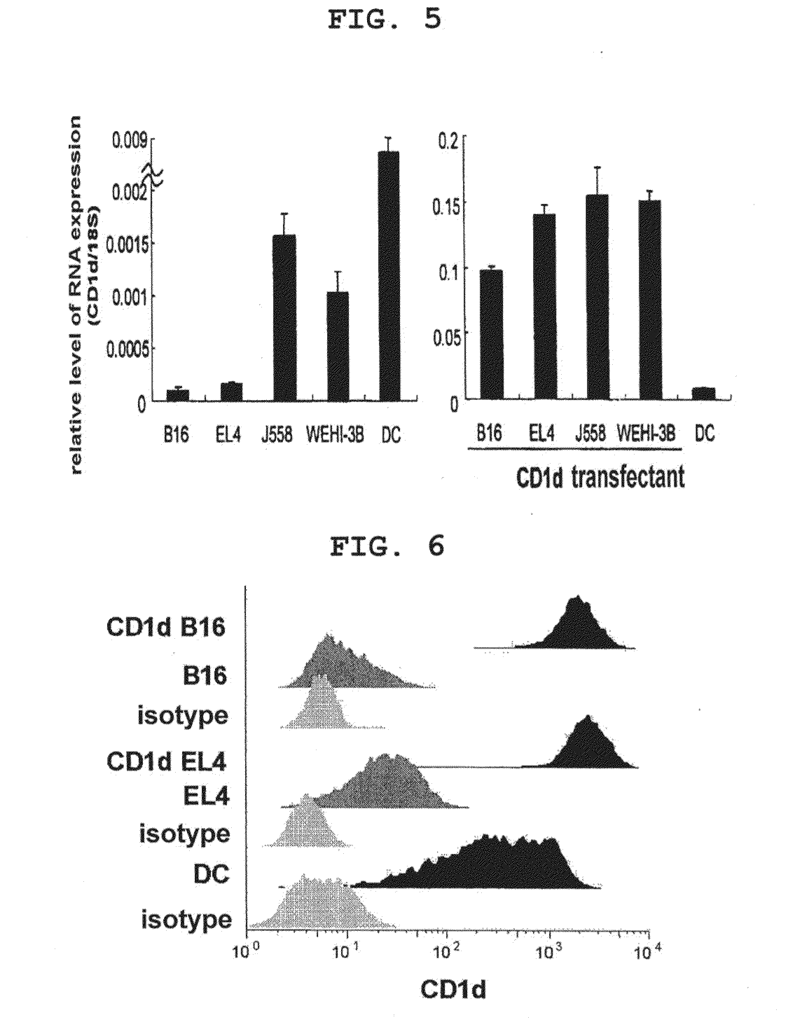

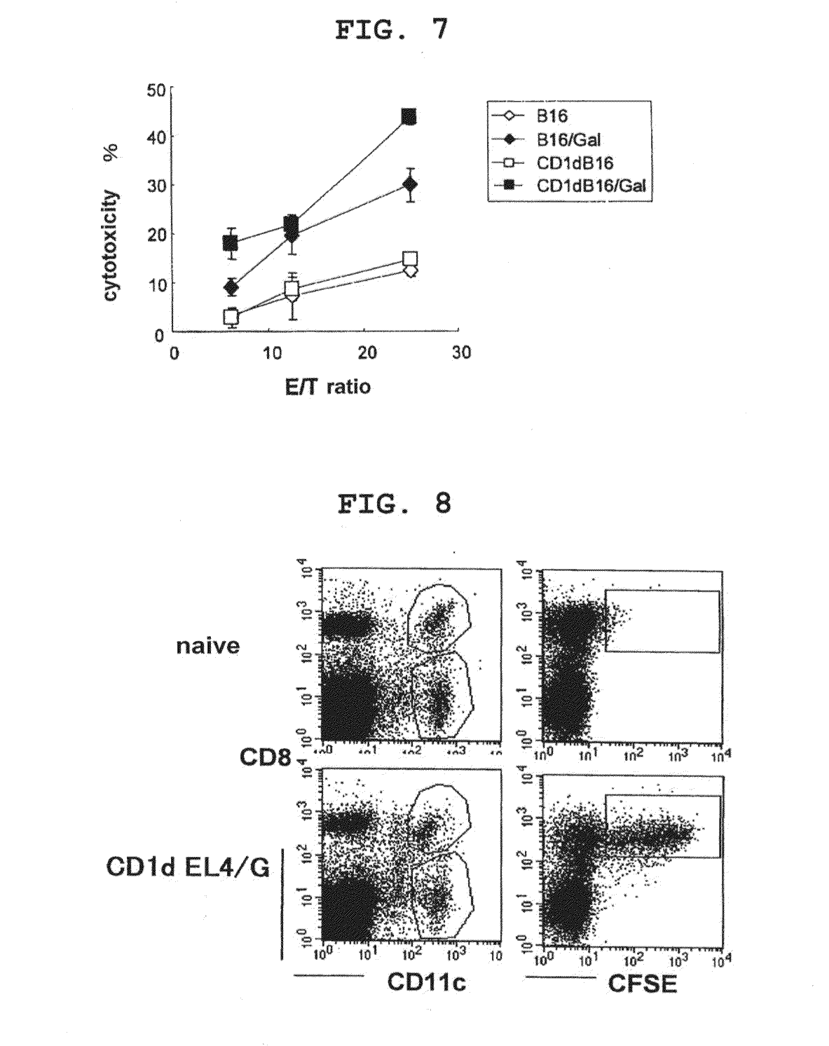

As a result, the expression of CD1d mRNA was confirmed in all tumor cell lines. The tumor cell lines into which the CD1d gene had been introduced showed a remarkable increase in the expression of CD1d mRNA (see FIG. 5).

Furthermore, the expression of CD1d protein was examined in B16 and EL4 cells that showed relatively lower levels of CD1d mRNA expression. The tumor cell lines into which the CD1d gene had been introduced showed remarkably enhanced expression levels of CD1d protein due to the CD1d transgene (see FIG. 6).

From the foregoing, it is clear that these tumor cells express CD1d protein and that the expression of CD1d protein is remarkably enhanced in a cell having an introduced CD1d gene.

### Example 7

This example demonstrates that a CD1d-expressing tumor cell is a target of activated NKT cells.

Whether or not a CD1d-expressing tumor cell can be a target of activated NKT cells was considered. A tumor cell was pulsed with α-GalCer for 48 hr and labeled with ⁵¹Cr. After washing, the obtained tumor cell was co-cultured with liver-derived mononuclear cells and the amount of released ⁵¹Cr was measured. Based on this, the cytotoxic activity was measured.

As a result, a tumor cell that forcibly expresses CD1d pulsed with α-GalCer released a higher amount of ⁵¹Cr than a tumor cell not pulsed with α-GalCer. This shows that a tumor cell pulsed with a CD1d ligand activates NKT cells, and then is killed as a target of the NKT cells.

From the foregoing, it is clear that a cell pulsed with a CD1d ligand can be a target of NKT cells.

### Example 8

This example demonstrates that a CD1d-expressing tumor cell is a target of activated NKT cells.

EL4 cells that forcibly express CD1d pulsed with α-GalCer were labeled with CFSE (carboxyfluorescein succinimidyl ester) and administered to a mouse. After 10 hr, splenocytes were collected from the mouse, and CFSE uptake by dendritic cells was measured using a flow cytometer.

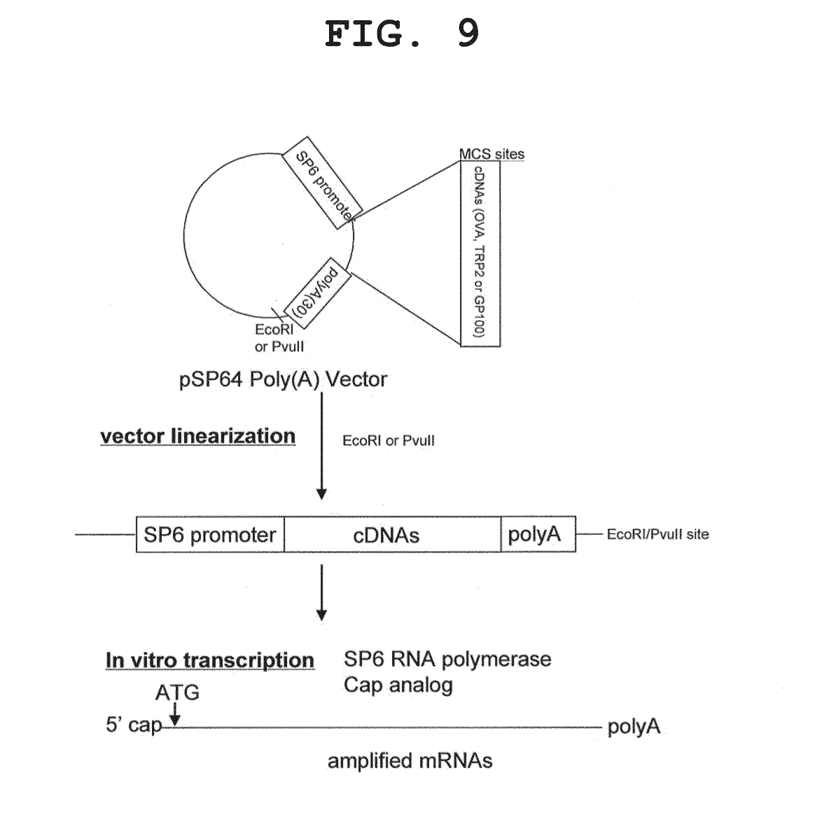

As a result, it has been confirmed that CD11c⁺ (particularly, CD11c⁺CD8a⁺) dendritic cells uptake CFSE (see FIG. 8).

The foregoing suggests that the administered tumor cell is killed by activation of NK/NKT cells, and the antigen is captured and presented by the adjacent dendritic cell, whereby the antitumor immunity can be achieved.

### Example 9

This example describes the materials and methods for Examples 10-15.

Mouse and Cell Line

6- to 8-week-old pathogen-free C57BL/6(B6) mice were purchased from CLEA Japan (Tokyo), and B6 CD4^{-/-} and CD8^{-/-} female mice were purchased from Jackson Laboratory (Bar Harbor, Me.). OT-I TCR gene recombinant mice, CD11c-DTR/GFP mice, and Jα18^{-/-} mice were provided by Dr. Heath (Walter and Eliza Hall Institute, Victoria, Australia), Dr. Littman (New York University, New York, N.Y.) and Dr. Taniguchi (RIKEN), respectively. The above-mentioned mice were reared under particular pathogen-free conditions, and studies were performed according to the RIKEN guidelines. B16, EL4 and EG7 cell lines were obtained from the American Type Culture Collection (Rockville, Md.), and NIH3T3 cells were obtained from the RIKEN BANK. For transfection of CD1d, pMX-mCD1d-IRES-GFP containing mCD1d was transfected into B16 melanoma or NIH3T3 cells by retrovirus as described in J. Immunol., 178: 2853-2861 (2007). Then, based on the GFP expression, the cell was separated by an FACS Vantage Cell Sorter.

Cell Preparation

DC derived from bone marrow was prepared from a bone marrow precursor cell as described in J. Exp. Med., 176: 1693-1702 (1992). On day 6, α-GalCer (100 ng/mL) was added to DC for 40 hr, during which 100 ng/mL of LPS was added for the last 16 hr. For loading of α-GalCer on other cells, fibroblasts (NIH3T3 or CD1d^{hi}-NIH3T3) or tumor cells were cultured for 48 hr in the presence of 500 ng/mL α-GalCer. The cells loaded with a-GalCer were washed 3 times before injection. CD1d^{hi}-NIH3T3 was prepared as described in J. Immunol., 178: 2853-2861 (2007) and the like. CD70-NIH3T3, Rae1ε-NIH3T3, Rae1γ-NIH3T3, and Mult1-NIH3T3 were prepared as follows. Mouse CD70 complementary (c)DNA, Rae1ε cDNA, Rae1γ cDNA and Mult1 cDNA were cloned to retrovirus vectors having pMX-ligand cDNA-IRES-GFP and infected with NIH3T3. Then, the cells were sorted by GFP expression.

Preparation of EGFP, OVA and TRP-2 mRNA

Respective full-length cDNAs (EGFP, OVA, TRP-2) were subcloned into pSP64 poly(A) vectors (Promega, Madison, Wis.) (see FIG. 9). Vectors having respective cDNAs were amplified and linearized by digestion with enzyme EcoRI (for EGFP or OVA) and PvuII (for TRP-2). After preparation of capped mRNA, Ribo m⁷G cap analogs (Ambion, Austin, Tex.) were incorporated using RiboMax Large scale RNA large scale RNA production systems-SP6 (Promega) into Ribo Max transcription reaction to amplify mRNA (see FIG. 9).

Transfection of mRNA

RNA transcribed in vitro (IVT) was transfected into various cell lines using a TransMessenger transfection kit (Qiagen) according to the protocol of the manufacturer. One day before transfection, cells (2×10⁵) were seeded in a 60 mm tissue culture petri dish. The next day, the cells were washed 3 times with PBS and transfected with a different amount of IVT RNA. The ratio of mRNA, enhancer solution, and transmessenger reagent was 1:2:4. The cells were transfected at different times and directly harvested (2 hr, 4 hr, 8 hr or 16 hr) or replenished with RPMI 1640 containing 10% bovine serum albumin and cultured overnight (2 hr+16 hr culture, 4 hr+16 hr culture, 8 hr+16 hr culture or 16 hr+16 hr culture). These cells were analyzed by FACS or confocal laser scanning microscope (TCS-SP2 Leica DMRE, Heidelberg, Germany), or subjected to a measurement by ELISA (Morinaga).

Real Time PCR Assay

Using RNeasy kit (Qiagen, Valencia, Calif.) or Trizol reagent (Invitrogen, Carlsbad, Calif.), total RNAs were isolated from various cell lines according to the protocol of manufacturer. For isolation of total RNA from a small number of cells (less than 2×10⁵) with a Trizol reagent, 5 μg of glycogen (Roche, Indianapolis, Ind.) was used for co-precipitation. After synthesis of cDNA from 1 μg of total RNA, mRNA expression was quantified by real-time PCR using Taqman probe primer (Applied, Biosystems).

In Vitro Tumor Studies

NIH3T3 fibroblast (5×10⁵ cells/mouse) loaded with α-GalCer and transfected with an mRNA encoding antigen was intravenously injected to the mice to immunize them. In an experiment to evaluate the development of protective immunity against tumor administration, tumor cell was subcutaneously administered to immunized mouse 2 weeks later, and the tumor size was measured. In some experiments, CD4^{-/-} and CD8^{-/-} mice were used as recipient mice.

Statistical Analysis

Differences in the vitro data were analyzed by Mann-Whitney U-test wherein P<0.05 was considered statistically significant.

### Example 10

This example demonstrates that determination of optimal conditions for transfection of mRNA encoding antigen into allogeneic cells.

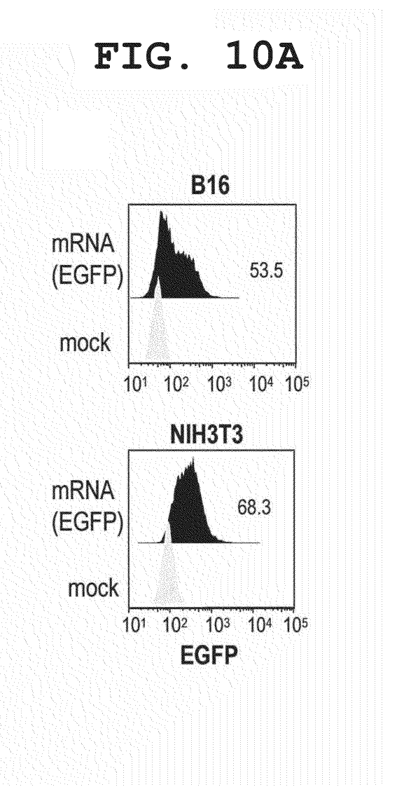

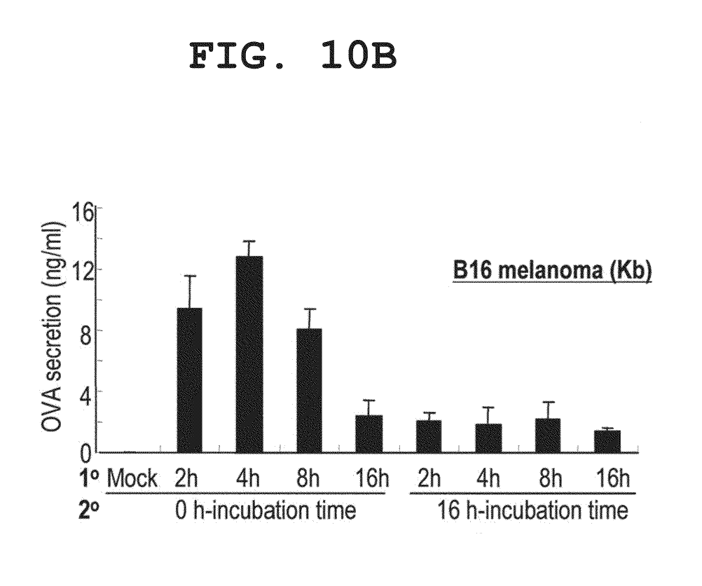

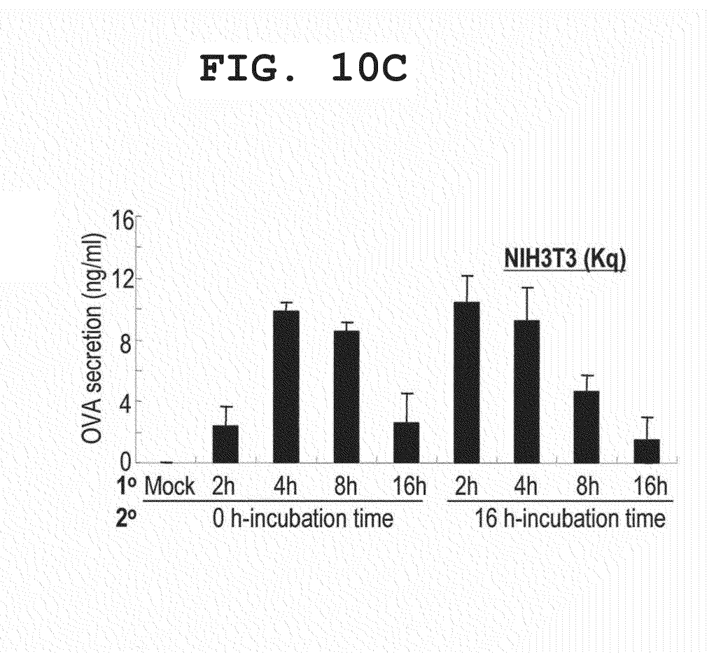

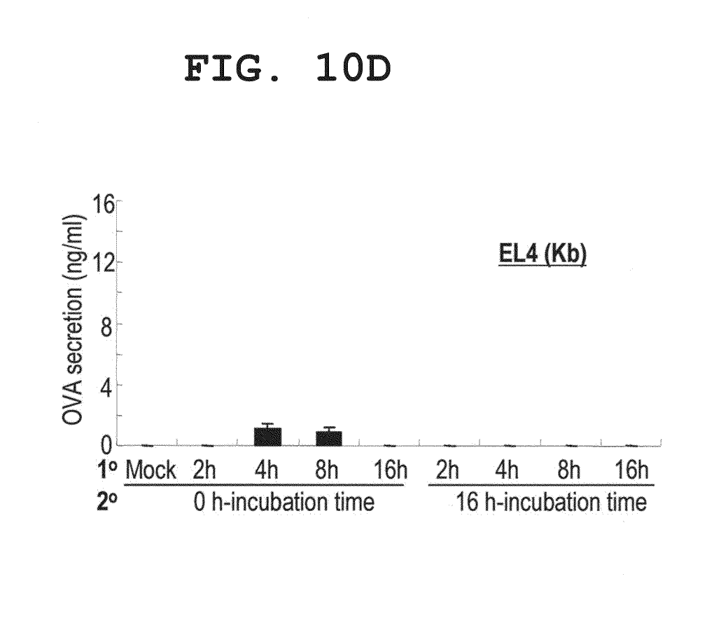

To determine the concentration-dependent transfection rate of an mRNA encoding an antigen into the cells, expression of EGFP mRNA, which was transcribed in vitro from linearized SP6 vector having EGFP, was evaluated. According to the mRNA concentration levels, EGFP expression in transfected B 16 melanoma cell (H2-K^{b}) or NIH3T3 fibroblast (H2-K^{q}) was analyzed by fluorescence microscopy. By comparison of transfection at different mRNA concentrations, 5 μg of EGFP mRNA was determined to be sufficient for the expression of EGFP in B16 cells and NIH3T3 cells. EGFP was sufficiently expressed in the both cells at hour 4, which continued at least for 12 hr. As a result of FACS analysis, transfection efficiency of EGFP mRNA into B16 melanoma cell or NIH3T3 fibroblast was almost the same (see FIG. 10A; data is of representative example of 3 independent experiments). It was far superior to the transfection efficiency (less than 5%; data not shown) into EL4 thymoma cell (H2-K^{b}).

Then, the time when the protein production reaches maximum after mRNA transfection was determined. The OVA protein level produced by B16, EL4, or NIH3T3 cells transfected with 5 μg of OVA mRNA (indicated as B16-ova, EL4-ova and NIH3T3-ova, respectively) was measured by ELISA after cell lysis. By evaluation of transfection time (2-16 hr), B16-ova and NIH3T3-ova were found to produce the highest level of OVA protein at 4 hr after transfection.

Whether the cell continues to produce OVA protein after transfection was analyzed by the measurement of OVA protein level. As shown in FIGS. 10B and 10C, expression of OVA protein by NIH3T3 cells transfected with OVA mRNA was of the same level as the B16 transfectant; however, it continued for a longer time than the B16 transfectant. An EL4 cell line transfected with OVA mRNA showed a low transfection level and scarcely expressed OVA protein (see FIG. 10D). Thus, NIH3T3 fibroblasts were selected for the subsequent experiments.

### Example 11

This example demonstrates transfection of the CD1d gene into a cell line without a co-stimulatory molecule.

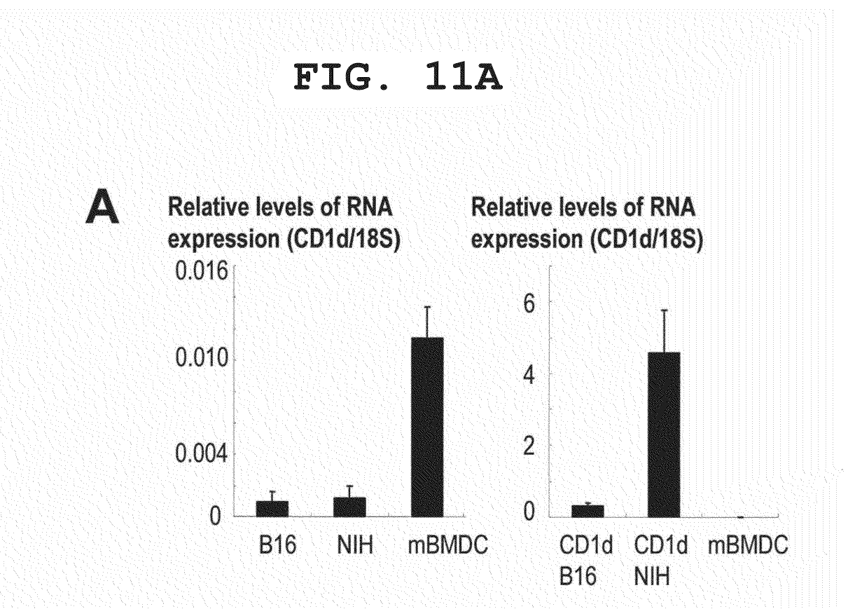

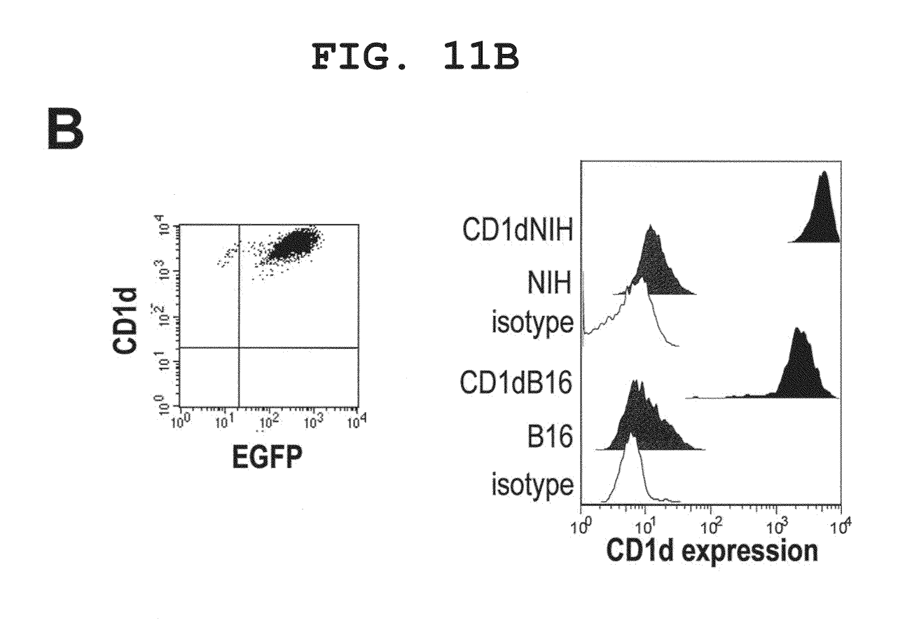

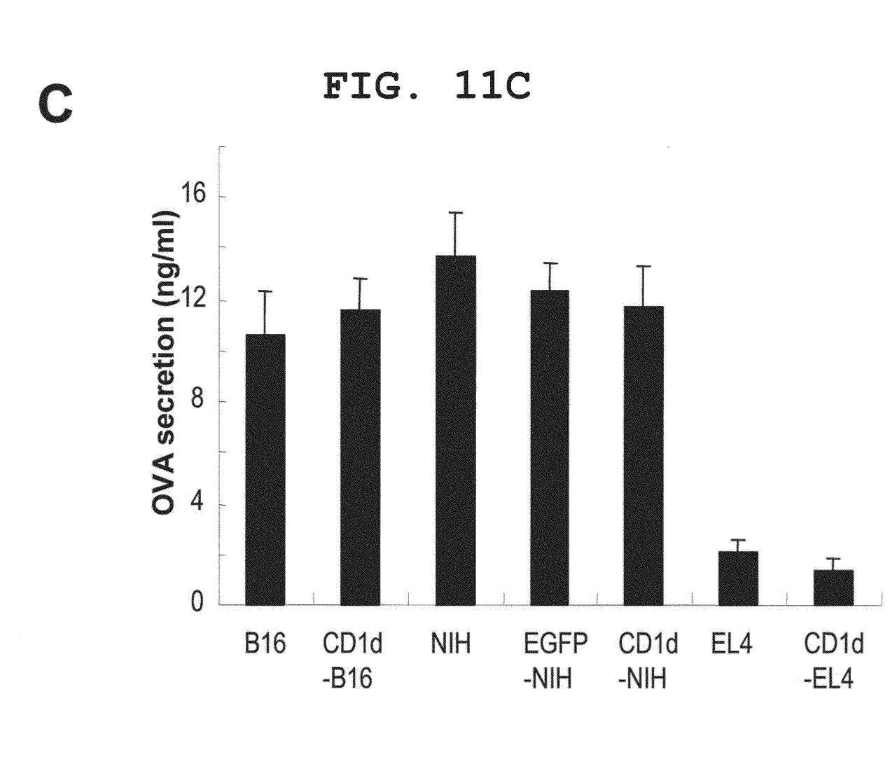

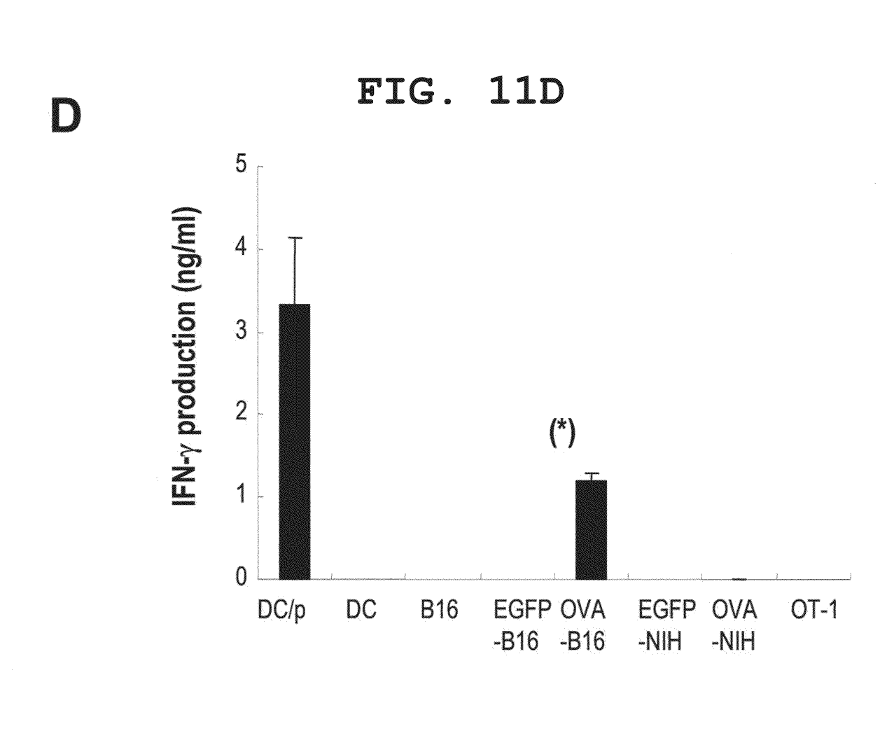

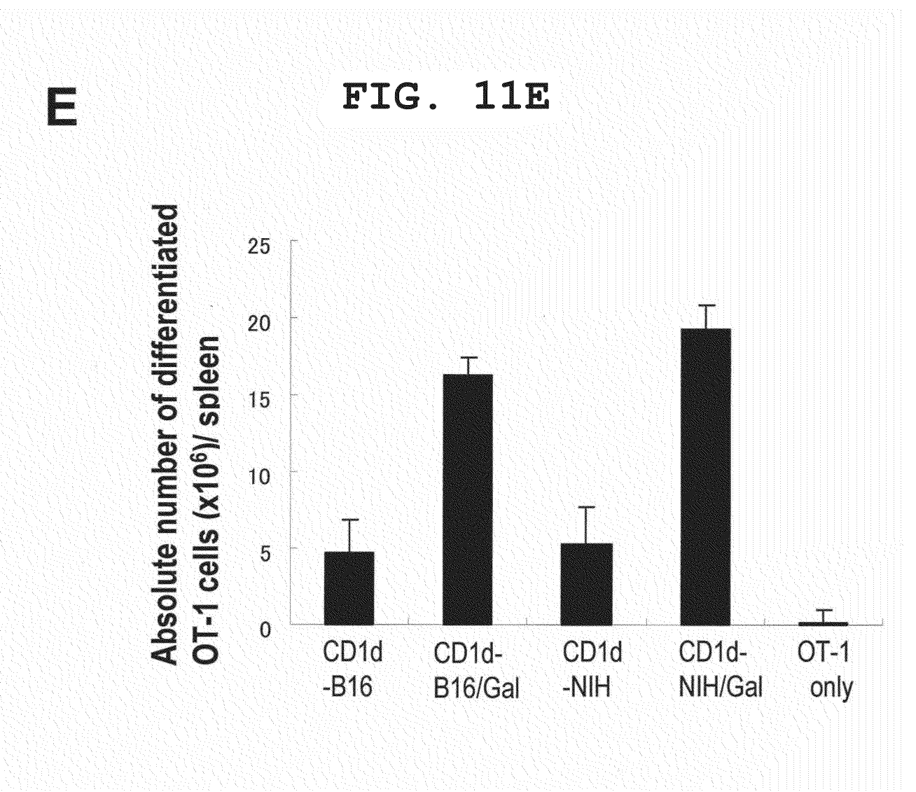

A tumor cell that expressed the CD1d molecule can present α-GalCer on primary iNKT cells even if it does not have a costimulatory molecule. It was confirmed that NIH3T3 fibroblast and B16 melanoma cell do not express CD40, CD70, CD86 and MHC class II. The CD1d expression levels of parental cell lines NIH3T3 (NIH in the figures) and B16, as well as stable transfectants transduced (transfected) with retrovirus expressing high level mouse CD1d, were determined (see FIGS. 11A and 11B). Stable CD1d^{hi} cell lines (CD1dNIH, CD1dB16, respectively, in the figures) were selected at a purity of >98% by sorting using FACS Vantage Cell Sorter (see FIG. 11B, left). The parental cells of B16 melanoma and NIH3T3 showed a lower CD1d expression level than bone marrow-derived DC (mBMDC in the figures) (see FIG. 11A). The CD1d expression levels of the cell lines and DC were compared by real-time PCR, and CD1d^{hi}-NIH3T3 cell (CD1dNIH in the figures) was found to have the highest level. This finding was also confirmed by FACS (see FIG. 11B, right).

To measure the expression of MHC class I antigen peptide in cell lines transfected with mRNA, the following steps were taken. An established cell line that expresses CD1d at a high level or a low level was transfected with OVA mRNA and, 4 hr later, an OVA expression level of the cell lysate was analyzed by ELISA. It was found that the OVA expression level of B 16 parental cell and CD1d^{hi}-B16 transfectant (B16, CD1d-B16, respectively, in the figures) was almost the same as that of NIH3T3 or CD1d^{hi}-NIH3T3 transfectant (NIH, CD1d-NIH, respectively, in the figure) (see FIG. 11C). It was confirmed that not only a cell line but also a transfectant containing EGFP-NIH3T3 (EGFP-NIH in the figures) or CD1d^{hi}-NIH3T3 (CD1d-NIH in the figures) could be stably transfected with mRNA.

A direct presentation activity of each transfectant as an antigen presenting cell was measured. To easily analyze OVA specific T cell response in vitro, B16 cell line highly expressing class I was established by exposure to recombinant IFN-γ for 12 hr. The parental cell or transfected cell was co-cultured with CD8⁺ T cells with recombined OVA specific TCR (OT-I cell, OT-1 in the figures) for 48 hr, and the IFN-γ level of the supernatant was measured. Secretion of IFN-γ increased by the supernatant derived from the B16 cell transfected with OVA-mRNA (B16-ova; OVA-B16 in the figures). However, such increase was not seen in the case of NIH3T3 transfected with OVA-mRNA (NIH3T3-ova; OVA-NIH in the figures) even though it secreted OVA protein at the same level as in the B16 cell transtected with OVA-mRNA (see FIG. 11D). This means that OVA peptide expresses in relation to MHC class I molecule as in OT-I and B16(K^{b}), but is a mismatch with NIH3T3 cell (K^{q}).

As shown in FIG. 11D, despite the same level of OVA secretion, OT-I cell did not recognize peptide antigen on NIH3T3 which is mismatch with MHC class I. To measure the in vivo antigen presenting ability of a fibroblast transfectant, OVA-mRNA transfectant loaded or not loaded with α-GalCer was given to mouse injected with OT-I cell. The absolute number of divided OT-I cells in the immunized mouse was analyzed 3 days later. As shown in FIG. 11E, OT-I cells of the mouse given CD1d^{hi}-NIH3T3 loaded with α-GalCer and transfected with OVA-mRNA (CD1d^{hi}-NIH3T3/Gal-ova; CD1d-NIH/Gal in the Figure) grew more than the OT-I cells of the mouse given CD1d^{hi}-NIH3T3 transfected with OVA-mRNA (CD1d^{hi}-NIH3T3-ova; CD1d-NIH in the figures). The number of OT-I cells of the mouse given CD1d^{hi}-NIH3T3/Gal-ova was the same as that of the mouse given tumor/Gal, i.e., CD1d^{hi}-B16/Gal-ova (CD1d-B16/Gal in the figures) (see FIG. 11E; data shows a representative example of two independent experiments using 2 mice per group, and the difference of CD1d^{hi}-NIH3T3-ova vs CD1d^{hi}-NIH3T3/Gal-ova and CD1d^{hi}-B16-ova (CD1d-B16/Gal in the figures) vs CD1d^{hi}-B16/Gal-ova was p<0.05 (significant). Thus, CD1d^{hi}-NIH3T3-ova cannot stimulate OT-I cell in vitro due to the MHC class I mismatch (see FIG. 11D). However, CD1d^{hi}-NIH3T3/Gal-ova could grow OT-I cell in vivo. Since it grows in this way despite the MHC class I mismatch, cross presentation by endogeneous DC in an allogeneic host is suggested.

### Example 12

This example demonstrates that fibroblasts loaded with α-GalCer activates allogeneic NK and iNKT cells in vivo.

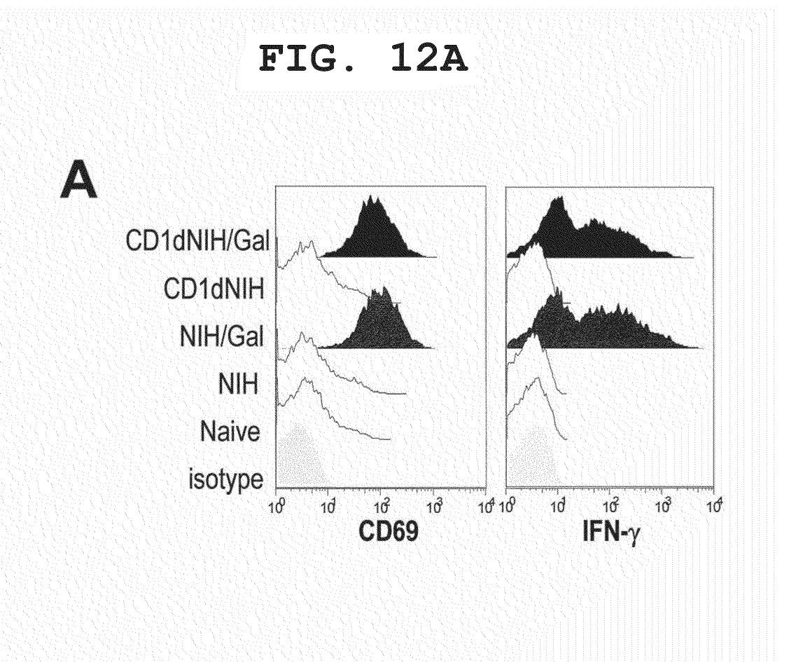

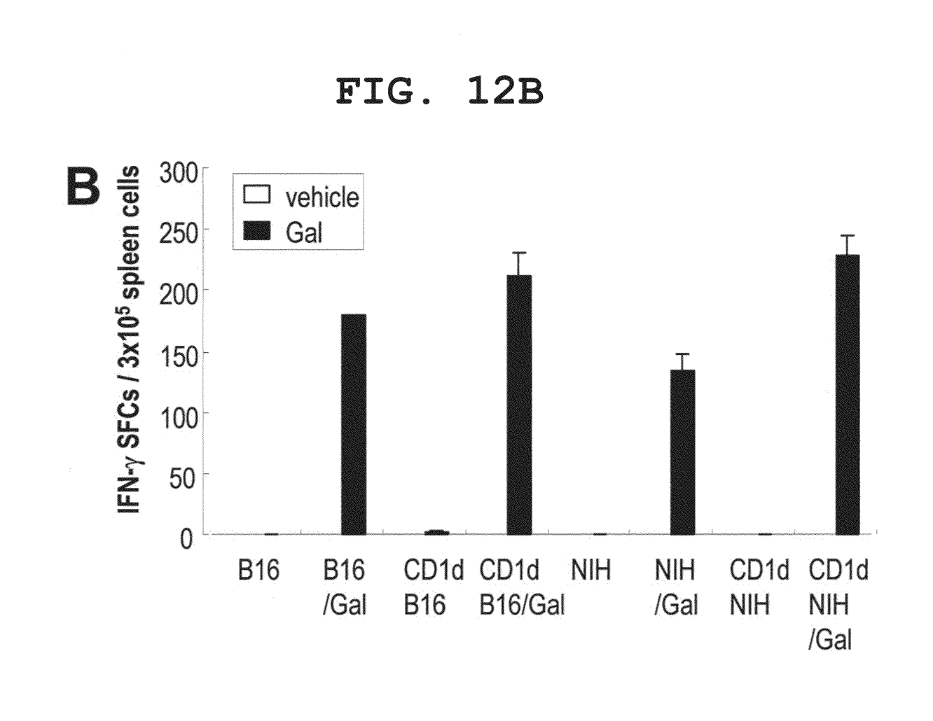

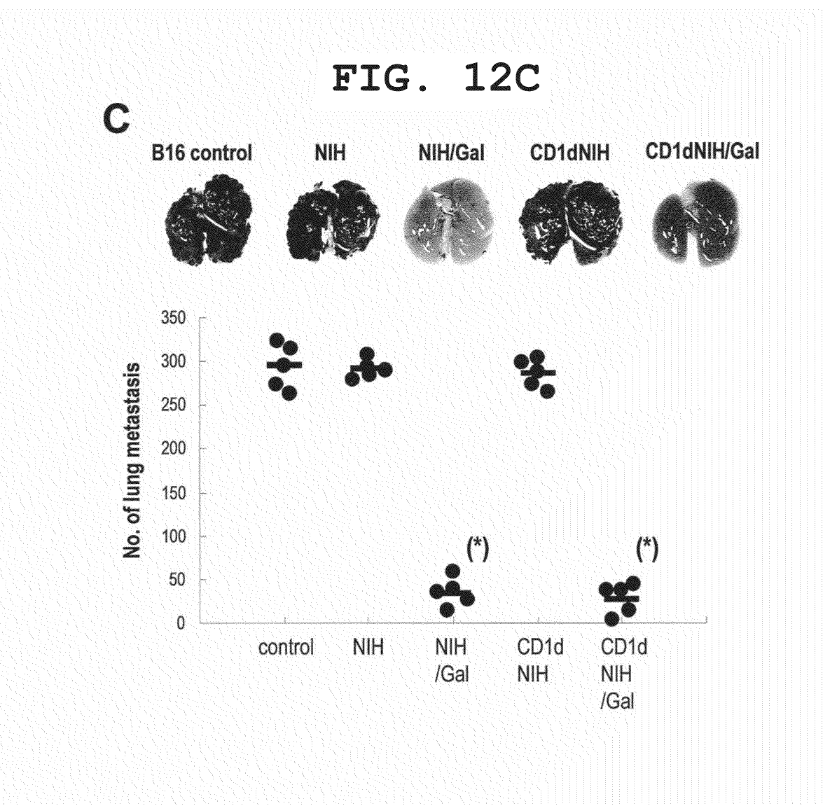

To examine whether allogeneic cells stimulate innate immunity system by α-GalCer in vivo, spleen cells of an immunized mouse were stained with CD3-FITC and NK1.1-APC, and the response of NK cells (CD3⁻NK1.1⁺) was analyzed by flow cytometry for expression of CD69 (stained with CD69-PE) and IFN-γ (stained with IFN-γ-PE) at 16 hr from immunization (see FIG. 12A). In the mouse given CD1d^{hi}-NIH3T3/Gal (CD1dNIH/Gal in the figures), NK cells increased CD69 expression and secreted IFN-γ. The NK cells of the mouse injected with NIH3T3 (NIH in the figures) or CD1d^{hi}-NIH3T3 (CD1dNIH in the figures) showed only a weak allogeneic response.

To analyze whether the parental cell (NIH3T3 or B16 cell) transfected with CD1d activates iNK cell by α-GalCer, spleen cells 2 days after immunization were suspended in the presence (see FIG. 12B black) or absence (see FIG. 12B white) of 100 ng/mL α-GalCer to re-stimulate the cells in IFN-γ ELISPOT assay. The number of IFN-γ producing spots of a mouse cell injected with NIH3T3/Gal (NIH/Gal in the figurea) or CD1d^{hi}-NIH3T3/Gal (CD1dNIH/Gal in the figures) was similar to that of B16/Gal and CD1d^{hi}-B16/Gal (CD1B16/Gal in the figures), respectively. The data show average of three mice per group. This suggests that CD1d^{hi}-NIH3T3/Gal and CD1d^{hi}-B16/Gal act as antigen presenting cells for the innate immune responses of iNKT cells and subsequent NK cell responses.

The antitumor effect by allogeneic fibroblast transfected with mRNA and loaded with CD1d ligand was examined using B16 lung metastasis models. 2×10⁵ cells of B16 (control) were uniformly administered, 3 hr later, NIH3T3 or CD1d expression enhanced NIH3T3 (CD1d^{hi}-NIH3T3), or NIH3T3 loaded with α-GalCer (NIH3T3/Gal) or CD1d expression-enhanced NIH3T3 (CD1d^{hi}-NIH3T3/Gal) (each 5×10⁵ cells) were intravenously administered to the mice of each group. Then, after 14 days from the administration, the lung was removed from each mouse, and the antitumor effect was evaluated (per group, n=5) (see FIG. 12C).

As a result, the model mice of the group administered NIH3T3 loaded with α-GalCer (NIH3T3/Gal; NIH/Gal in the figures) or CD1d expression-enhanced NIH3T3 (CD1d^{hi}-NIH3T3/Gal; CD1dNIH/Gal in the figures) showed a marked decrease in tumors as compared to other group. A similar effect was obtained in two independent experiments. (*) means that the difference between NIH3T3/Gal, CD1d^{hi}-NIH3T3/Gal and other groups, i.e., NIH3T3 (NIH in the figures), CD1d^{hi}-NIH3T3 (CD1dNIH in the figures) and control, is p<0.05 and significant.

From the foregoing, it is clear that allogeneic fibroblast transfected with mRNA and loaded with CD1d ligand shows an antitumor effect sufficient to prevent lung metastasis, by the activation of innate lymphocyte.

### Example 13

This example demonstrates the important role of in vivo DC maturation in response to allogeneic fibroblasts loaded with α-GalCer.

When tumor cells loaded with α-GalCer are injected to a mouse, T cell response is known to require maturation of host DC after trapping an antigen. As shown in FIGS. 12A and 12B, NIH3T3/Gal (NIH/Gal in the figures) clearly activated natural lymphocytes.

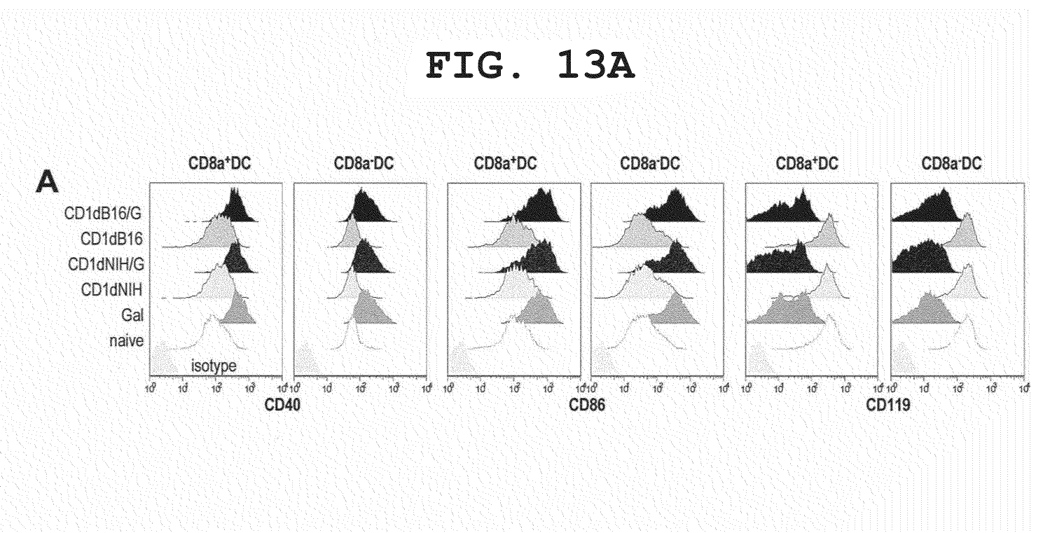

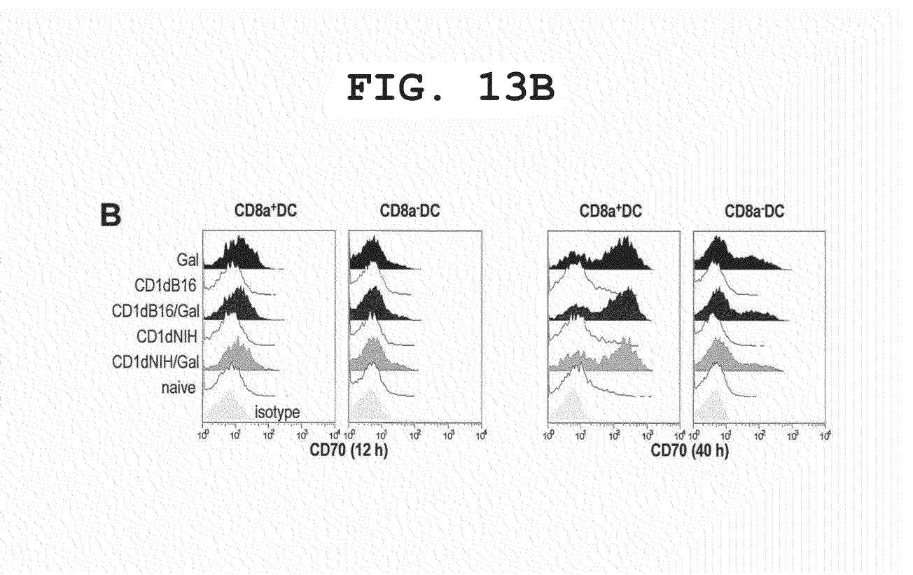

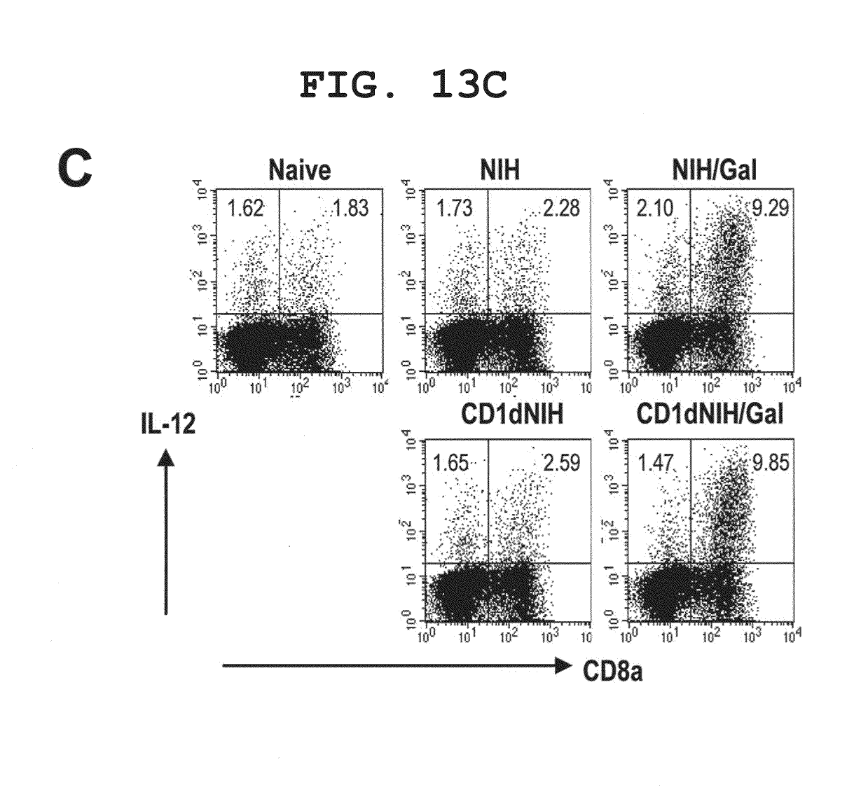

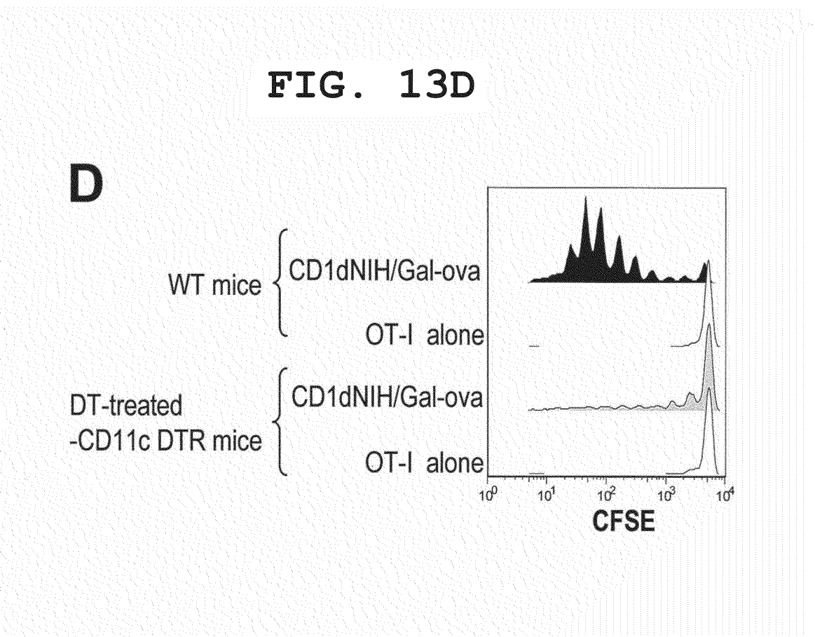

Next, it was determined whether DC maturation occurs in vivo after injection of NIH3T3 (NIH in the figures) or CD1d^{hi}-NIH3T3 (CD1dNIH in the figures), loaded or unloaded with α-GalCer, into the mouse (see FIG. 13A).

At 12 hr after the injection, spleen cells were collected, and expression of DC surface markers (CD40, CD86 and CD119) was analyzed by flow cytometry. Like the changes of DC maturation, it was found that the expression of CD40 and CD86 increased and expression of CD119 decreased. As shown in FIG. 13A, increase of CD86 expression in DC (CD8α⁺ and CD8α⁻ subset) was similar to the increase of expression found in a mouse immunized with CD1d^{hi}-B16/Gal (CD1dB16/Gal in the figures) or free α-GalCer. Since a recent report has documented that DC immunized by intravenous injection of free α-GalCer expresses CD70, CD70 expression was also analyzed similarly (see FIG. 13B). It was found that CD70 levels do not increase for 12 hr after injection of CD1d^{hi}-B16/Gal or CD1d^{hi}-NIH3T3/Gal (CD1dNIH/Gal in the figures) but increase in 40 hr. It was found that a greater amount of CD70 was expressed in CD8a⁺DC than CD8a⁻DC in later stages. As a sign of maturation of functional DC, IL-12 secretion can be generally mentioned. IL-12 secretion was also analyzed (see FIG. 13C).

DC derived from mice at 4 hr from immunization by intravenous injection of NIH3T3/Gal or CD1d^{hi}-NIH3T3/Gal secreted high level of IL-12, but DC derived from mice immunized with NIH3T3 cell or CD1d^{hi}-NIH3T3 did not. Since such DC maturation, modification of cell surface marker, and IL-12 secretion are not found in Jα18-deficient mouse, it was found that DC maturation essentially requires iNKT cells. These data suggest that DC starts maturation immediately after injection of allogeneic fibroblasts loaded with α-GalCer. Not only α-GalCer directly loaded on fibroblasts, but also α-GalCer trapped by a host DC, indirectly activates iNKT cells and mature DC.

Next it was determined that DC in the body are essential for inducing adaptive immunity in CD1d^{hi}-NIH3T3/Gal-ova injection mice.

In FIG. 11E, OT-I cells do not match with CD1d^{hi}-NIH3T3/Gal-ova (CD1dNIH/Gal in the figures) in class I in immunized mice. Therefore, to determine whether DC of the host is involved in the presentation of OVA antigen to OT-I cell in vivo, CD11c+DC was removed from the host by using CD11c-diphtheria toxin receptor (DTR) recombinant (CD11c-DTR/GFP) mice treated with diphtheria toxin (DT). In the mice, OT-I cells scarcely grew, and the role of DC in the cross presentation of antigen in the mice given a CD1d^{hi}-NIH3T3/Gal-ova cell was verified.

### Example 14

This example demonstrates the strong adaptive immune response afforded by immunization of C57BL/6 mouse with CD1d^{hi}-NIH3T3/Gal-ova.

When the inventive method of producing an immune response to fibroblasts transfected with mRNA is once established using a mouse injected with a gene recombinant OT-I cell, it becomes more important to achieve an immune response generated in a wild type mouse.

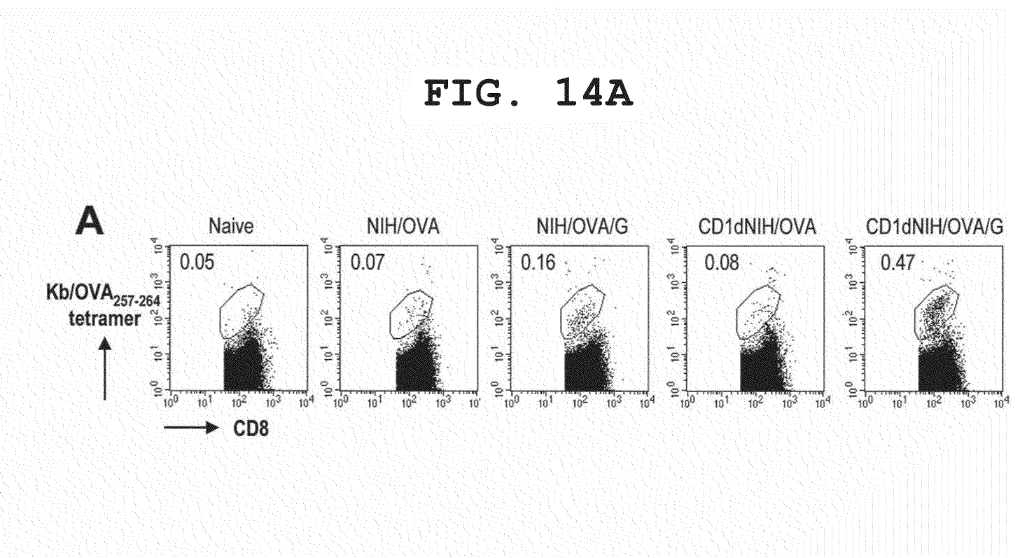

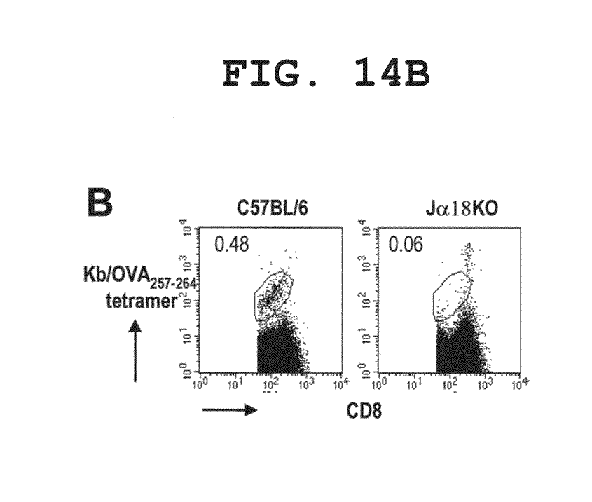

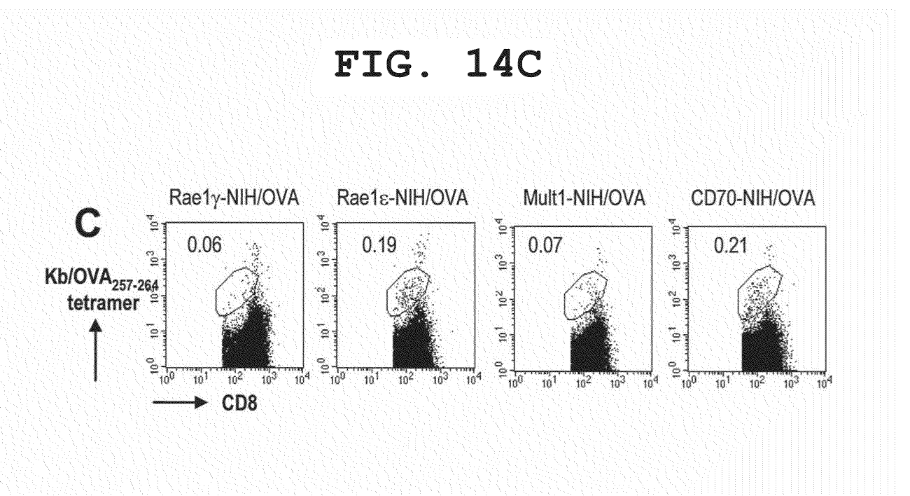

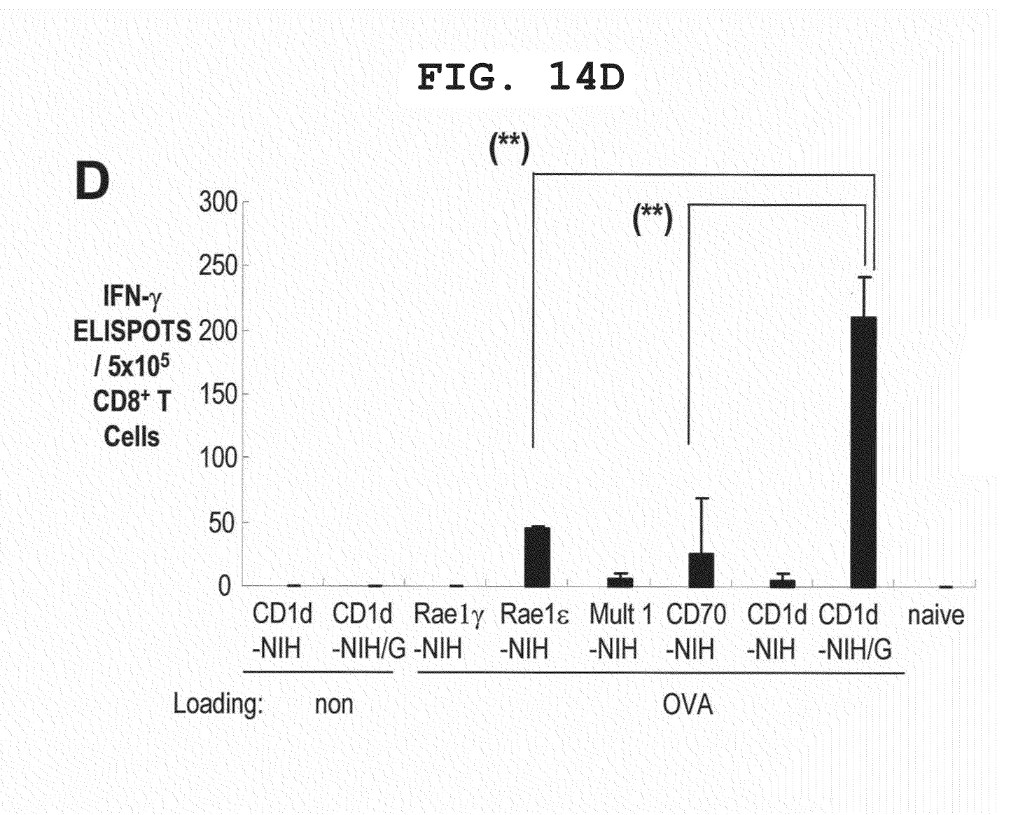

Next it was determined whether a wild-type mouse immunized with CD1d^{hi}-NIH3T3/Gal-ova acquires antigen specific T cell immunity (see FIG. 14A). In particular, it was determined whether activation of iNKT cells and CD1d expression level in cells having the antigen are important for the induction of acquired immunity. To perform this study, mice were immunized with various parental cells or CD1d^{hi}-NIH3T3 cells transfected with OVA mRNA: NIH3T3-ova (NIH/OVA in the figures), NIH3T3/Gal-ova (NIH/OVA/G in the figures), CD1d^{hi}-NIH3T3-ova (CD1dNIH/OVA in the figures) and CD1d^{hi}-NIH3T3/Gal-ova (CD1dNIH/OVA/G in the figures). After 7 days, spleen cells were collected and the number of CD8⁺ T cells specific to OVA peptide SIINFEKL (SEQ ID NO: 1) was analyzed by staining with K^{b}OVA₂₅₇₋₂₆₄ tetramer. As shown in FIG. 14A, the number of the cells positive to the OVA tetramer in the mouse given NIH3T3/Gal-ova or CD1d^{hi}-NIH3T3/Gal-ova was far higher than that of the same cell in the mouse given NIH3T3-ova or CD1d^{hi}-NIH3T3-ova. However, this did not occur in a Jα-18-deficient mouse (see FIG. 14B).

The level of T cell response after priming with CD1d ligand, α-GalCer, was compared with that after priming with an NK cell ligand such as retinoic acid early inducible-1ε (Rae1ε), Rae1γ, CD70, mouse UL16-binding protein-like transcript 1 (Mult1) and the like (see FIG. 14C). NK cell ligand was cloned by using a retrovirus vector having EGFP. The co-expression of each molecule and EGFP was confirmed by FACS analysis. T cell growth was evaluated by tetramer staining 1 week after immunization with CD70-NIH3T3-ova (CD70-NIH/OVA in the figures), Rae1ε-NIH3T3-ova (Rae1ε-NIH/OVA in the figures), Mult1-NIH3T3-ova (Mult1-NIH/OVA in the figures) or Rae1γ-NIH3T3-ova (Rae1γ-NIH/OVA in the figures). As shown in FIG. 14C, the group immunized with Rae1ε-NIH3T3-ova or CD70-NIH3T3-ova showed a K^{b}OVA₂₅₇₋₂₆₄ tetramer positive cell proliferation, but the group immunized with other NK ligands did not.

T cell response specific to OVA was also tested by using IFN-γ ELISPOT. The T cell response producing IFN-γ in mice given CD1d^{hi}-NIH3T3/Gal-ova was far higher than that in the mice given Rae1γ-NIH3T3-ova, Rae1ε-NIH3T3-ova, Mult1-NIH3T3-ova, CD70-NIH3T3-ova or CD1d^{hi}-NIH3T3-ova (see FIG. 14D). Thus, a fibroblast having the antigen and loaded with α-GalCer provides a stronger immune response by combining innate immunity and acquired immunity in naive mouse.

### Example 15

This example demonstrates induction of antitumor T cell activity by inoculation of CD1d^{hi}-NIH3T3/Gal-ova vaccine.

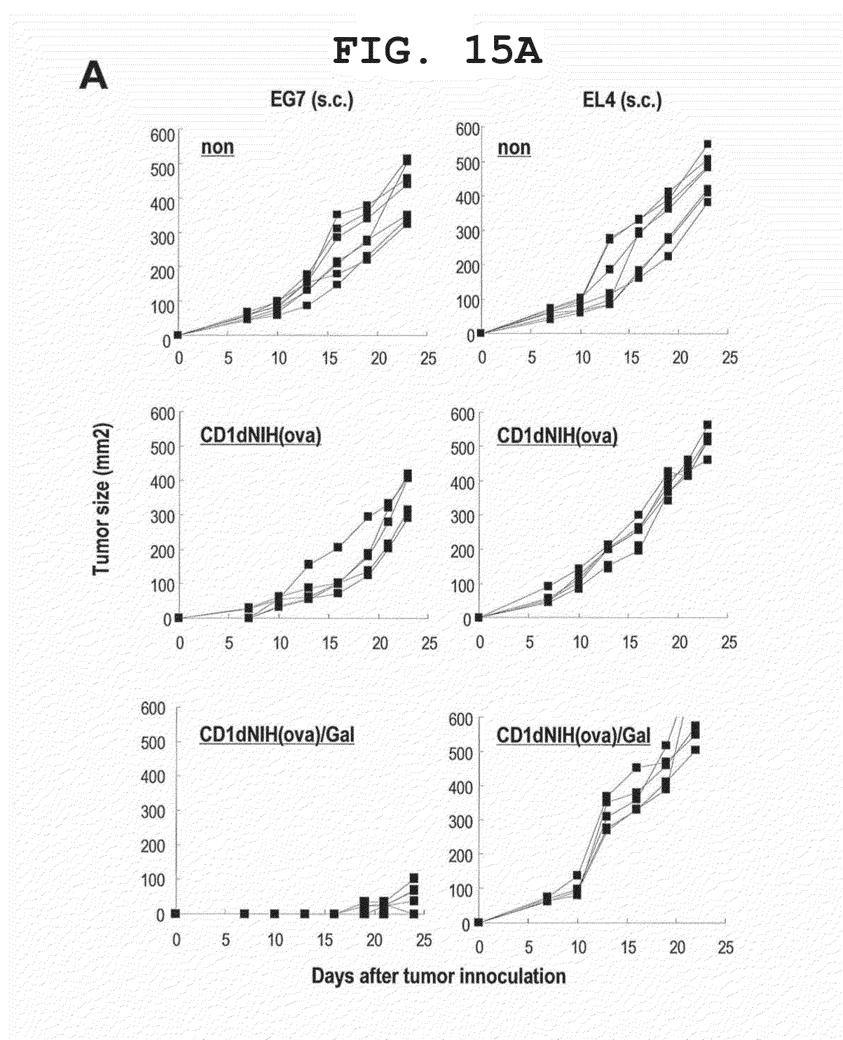

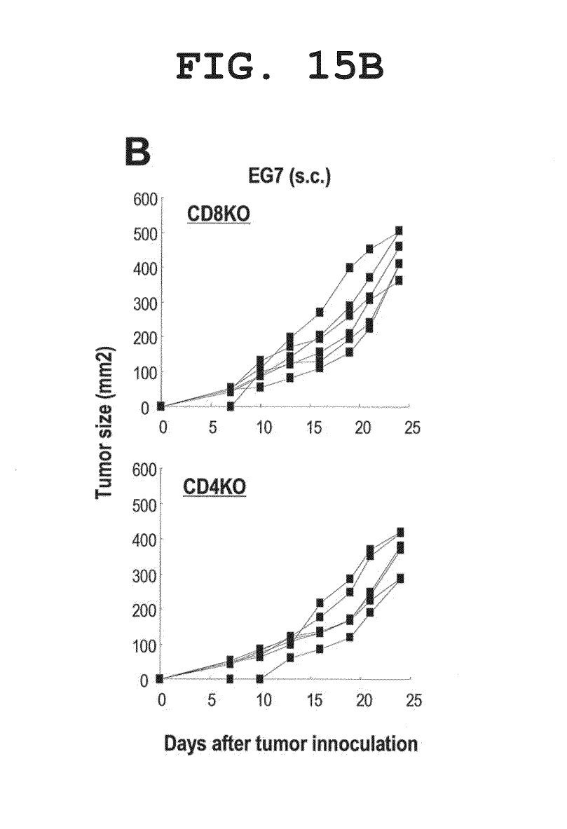

Whether T cell response in a mouse immunized with CD1d^{hi}-NIH3T3/Gal-ova can lead to the antitumor immunity was evaluated (see FIG. 15A). A mouse was immunized by intravenous administration of 5×10⁵ cell of CD1d^{hi}-NIH3T3-ova (CD1dNIH(OVA) in the figures) or CD1d^{hi}-NIH3T3/Gal-ova (CD1dNIH(OVA)/Gal in the figures) and, 2 weeks later, 1×10⁵ EL4 thymoma or OVA-expressing EL4 (EG7) was administered. The mouse administered CD1d^{hi}-NIH3T3/Gal-ova showed an antitumor effect on EG7 but not on EL4. This shows that the effect is a tumor specific immune response. The mouse given CD1d^{hi}-NIH3T3-ova (see FIG. 15A) or CD1d^{hi}-NIH3T3/Gal (data not shown) developed EL4 and EG7 tumors.

The defense against tumor development after intravenous inoculation requires CD4⁺ and CD8⁺T cell responses (see FIG. 15B). The tumor size was measured when it was shown on the graph (per group, n=6-8). Similar results were obtained in two independent experiments. Whether CD1d^{hi}-NIH3T3/Gal-ova cell similarly provides defense to tumor development after 30Gy irradiation was also tested, and similar results were also found in the group of mice subjected to irradiation (data not shown).

An OVA mouse model was immunized with an allogeneic cell line loaded with α-GalCer and transfected with mRNA, whereby the relationship between innate and acquired immunities was established (see FIGS. 15A-B).

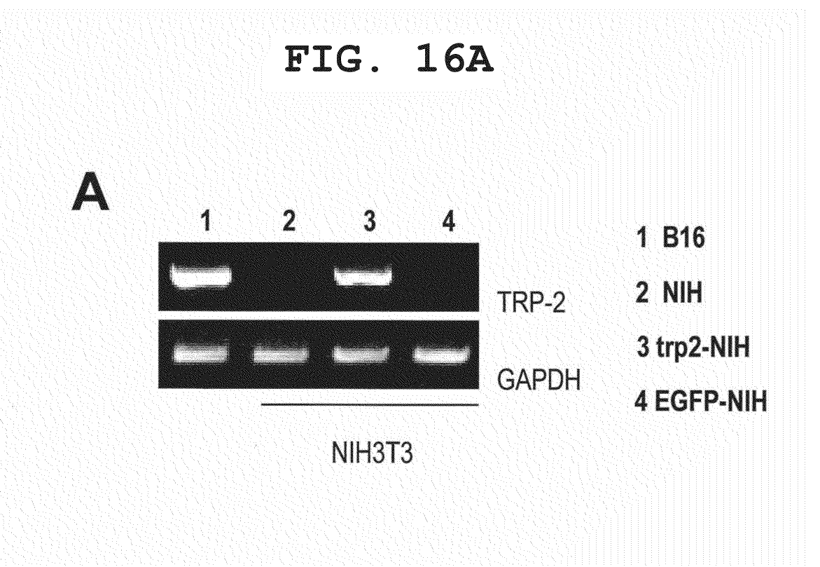

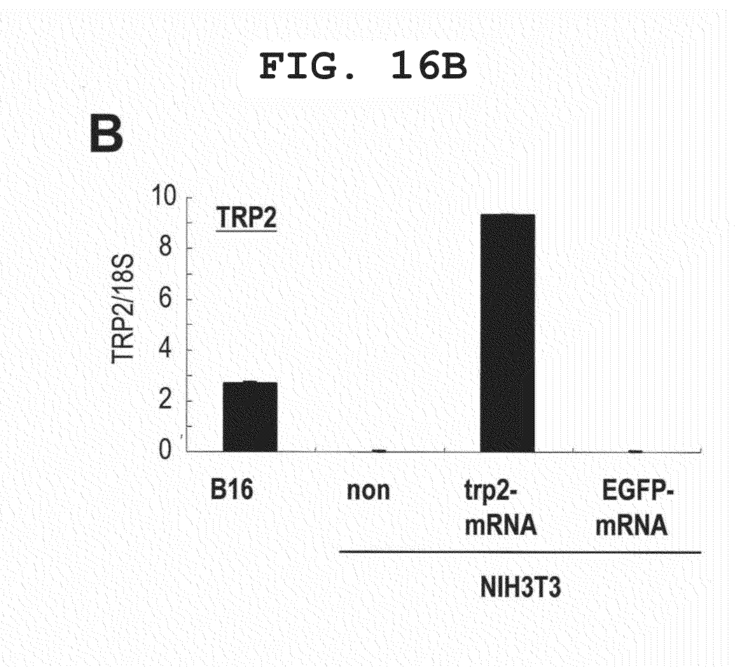

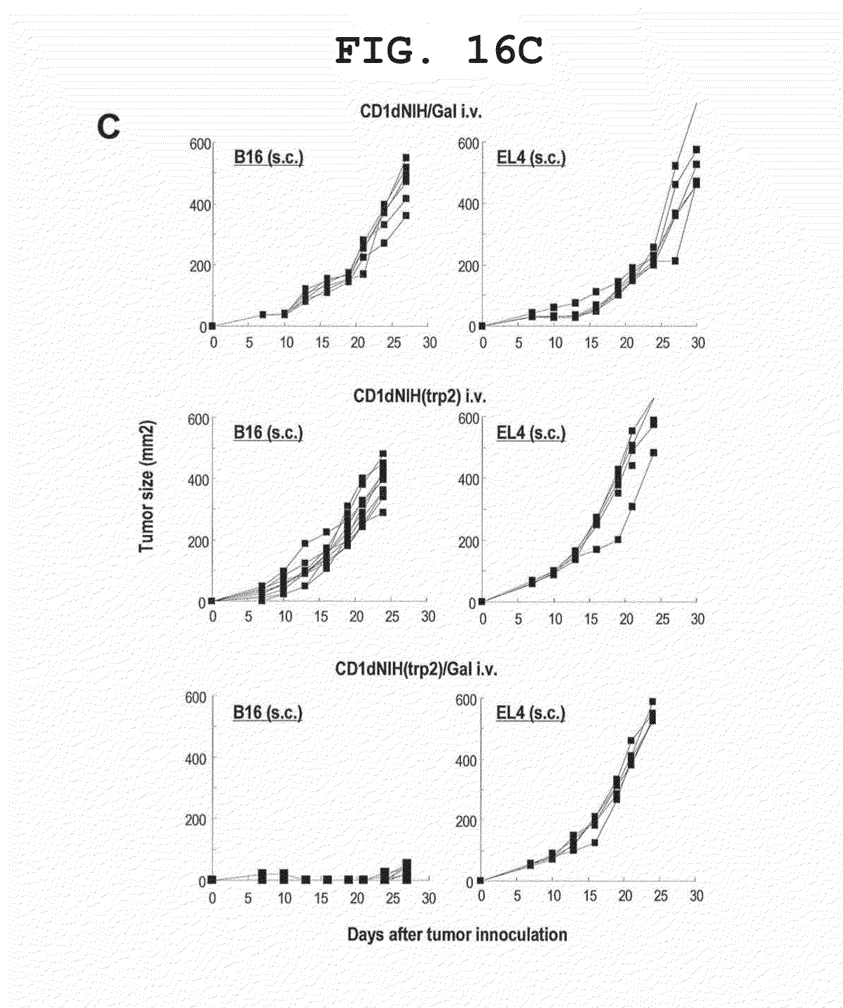

Then, the concept was applied to a real tumor model by immunizing the mouse with CD1d^{hi}-NIH3T3/Gal cell transfected with melanocyte differentiation antigen and tyrosinase-related protein 2 (trp2) mRNA (see FIGS. 16A and 16B). The trp2 expression in NIH3T3-trp2 (trp2-NIH in the figures) was confirmed by RT-PCR (see FIG. 16A), and confirmed by real-time PCR (see FIG. 16B). The results show that the expression is nearly three times that of trp2 endogenously expressed in B16 melanoma cell.

The adaptive antitumor response to injected CD1d^{hi}-NIH3T3/Gal transfected with mRNA encoding trp2 was evaluated (see FIG. 16C). Mice were immunized by intravenous administration of CD1d^{hi}-NIH3T3/Gal-trp2 (CD1dNIH(trp2)/Gal in the figures), CD1d^{hi}-NIH3T3/Gal (CD1dNIH/Gal in the figures) or CD1d^{hi}-NIH3T3-trp2 (CD1dNIH(trp2) in the figures). When B16 melanoma cells (5×10⁴) were administered to the mice 2 weeks later for the evaluation of antitumor defense, the mice administered with CD1d^{hi}-NIH3T3/Gal-trp2 (FIG. 16C lower left) showed inhibition of B16 tumor growth, but the mice administered with CD1d^{hi}-NIH3T3-trp2 (FIG. 16C middle left) or CD1d^{hi}-NIH3T3/Gal (FIG. 16C upper left) showed otherwise. None of the immunized mouse groups showed an antitumor immunity against EL4 thymoma cell (1×10⁵) (see FIG. 16C right). The tumor size was measured at the time point when it was shown on the graph (per group, n=6-8). Similar results were obtained in two independent experiments.

### Example 16

This example demonstrates that artificial adjuvant vector cells (aAVCs) activate iNKT cells in vivo in canine models.

aAVCs were prepared as follows. CD1d-HEK293 cells were cultured for 48 hours in the presence of 500 ng/mL of α-GalCer and then washed three times before transfection. Target antigen RNAs were transfected into CD1d-HEK293 cells using TransMessenger transfection kit (QIAGEN) following the manufacturer's instructions. Briefly, the ratio of mRNA, enhancer solution and transmessenger reagent was 1:2:4 for performing lipofection. α-GalCer-loaded CD1d-HEK293 cells were transfected for 4 hours and then cultures were replenished with DMEM containing 10% fetal bovine serum for 2 hours for OVA mRNA and 20 hours for EGFP or MART-1 mRNA. Transfected cells were analyzed by ELSIA for OVA and by flow cytometry for EGFP. To quantify protein by western blot analysis, 2×106 aAVC-MART-1 cells were lysed in 150 μL of sample buffer. Anti-MART-1 Ab-3 (NeoMarkers, Fremont, Calif.) and anti-mouse IgG HRP (Sigma-Aldrich, St. Louis, Mo.) were used for detection, and protein expression was measured by a luminescence image analyzer, LAS 1000 (FujiFilm Co, Tokyo, Japan).

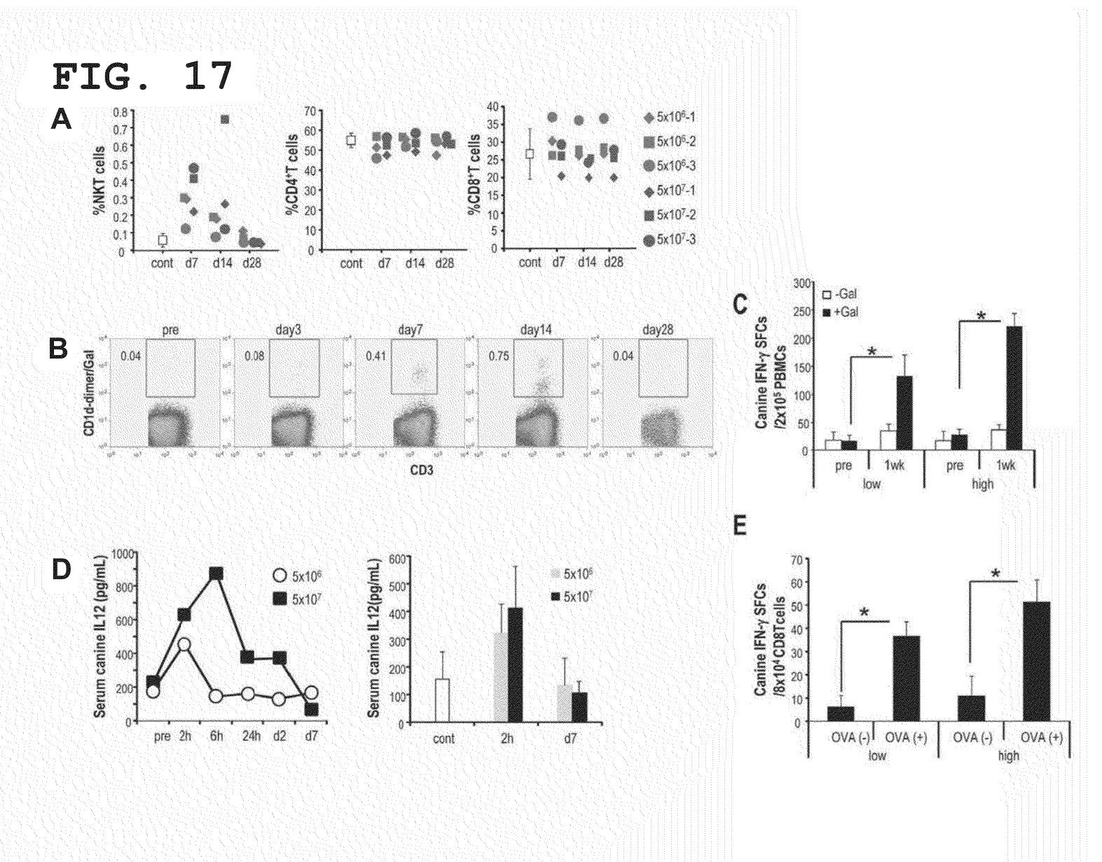

aAVCs were evaluated as potential vaccines in a preclinical safety and adverse event monitoring study using beagles. In these studies, 30 Gy-irradiated aAVCs were used. Two doses of aAVCs were given to 3 dogs per group with a low dose consisting of 5×10⁶ cells and a high dose of 5×10⁷ cells. The numbers of PBMC iNKT, CD4⁺ T and CD8⁺ T cells in the recipients were monitored by flow cytometry. The frequency of CD4⁺ T and CD8⁺ T cells did not change over 28 days of monitoring (see FIG. 17A middle and right panel).

It was previously reported that canine iNKT cells could be detected using mouse CD1d-dimer/α-GalCer (see, e.g., Yasuda et al., Vet. Immunol. Immunopathol., 132: 224-231 (2009)). The number of canine iNKT cells is generally lower than that in humans and mice, which was verified in the current study. The frequency of iNKT cells in the beagles was 0.018±0.009% of the total lymphocyte population in peripheral blood, i.e., 0.036±0.018% of CD3+ T cells. Therefore, a previously established method for detecting low numbers of iNKT cells in human cancer patients by coculturing PBMCs with 100 ng/mL α-GalCer-loaded murine DCs was used. Using this approach, canine iNKT cells could be detected and their kinetics followed after the injection of aAVCs (see FIGS. 17A left and 17B). The number of iNKT cells increased from day 7 to day 14, but went back to control level one month later (see FIGS. 17A left and 17B).

iNKT cell activation was also evaluated in aAVC-treated dogs using an IFN-γ ELISPOT assay (see FIG. 17C) (see Fujii et al; Nat. Immunol., 3: 867-874 (2002); Hermans et al., J. Immunol., 171: 5140-5147 (2003); and Shimizu et al., J. Immunol., 178: 2853-2861 (2007)). The number of IFN-γ producing cells in aAVCs-ova immunized dogs increased at 1 week after both the low and high dose aAVC treatment. These data further indicate that aAVCs stimulate iNKT cell proliferation. Most importantly, all dogs in groups receiving both doses of aAVC were monitored from the time of aAVC immunization until 1 to 4 weeks post-immunization, and no adverse effects were noted (see Table 1).

TABLE 1
Analysis of adverse events in canine.

| | | Pre | 1 week | 4 week |
|---|---|---|---|---|
| BUN (mg/dL) | Low | 18.5 + 2.5 | 19.2 + 5.7 | 17.3 + 1.1 |
| | High | | 15.6 + 4.3 | 20.2 + 4.3 |
| Cre (mg/dL) | Low | 0.65 + 0.14 | 0.67 + 0.05 | 0.6 |
| | High | | 0.60 + 0.14 | 0.57 + 0.12 |
| AST (U/L) | Low | 26.8 + 3.6 | 29.7 + 2.4 | 27.7 + 2.4 |
| | High | | 28.3 + 0.9 | 24.7 + 1.2 |
| ALT (U/L) | Low | 47.2 + 15.4 | 41.3 + 3.8 | 37.3 + 7.8 |
| | High | | 56.7 + 6.6 | 56.0 + 14.5 |
| CRP (mg/dL) | Low | 0.86 + 1.51 | 0.08 + 0.12 | 0.13 + 0.12 |
| | High | | 0.37 + 0.41 | 0.23 + 0.16 |
| ANA& antiviral Ab | Low | N.D. | N.D. | N.D. |
| | High | | N.D. | N.D. |

ANA: antinuclear antibody
N.D.: not detected

The safety of this therapy in the dog after three injections of aAVCs was verified (data not shown).

Whether antigen-specific T cell immunity can be generated in dogs after immunization with aAVC-ova also was tested. Serum was collected from the dogs at different time points after immunization with aAVC-ova, and IL-12 levels were found to be elevated 2 and 6 hours after treatment, suggesting that DC maturation in situ occurs early after aAVC immunization (see FIG. 17D). Fourteen days after an immunization, PBMC from immunized dogs were restimulated with or without OVA protein-transduced canine DCs for 36 hours and IFN-γ secretion by ELISPOT was measured. The number of OVA-specific IFN-γ secreting CD8+T cells was elevated with both doses of aAVCs (see FIG. 17E). Thus, this preclinical study demonstrated that aAVCs could safely and effectively generate an antigen-specific immune response in dogs.

### CONCLUSION

The present invention can induce T cell capable of specifically eradicating the tumor cells and pathogen-infected cells by preparing and utilizing mRNA characteristically expressed therein. Therefore, the present invention is highly useful for the establishment of a highly effective immunotherapy for diseases caused thereby. According to the present invention, moreover, the possibility of performing an order-made immunotherapy for individuals is created by clarifying the antigen property of the cell to be eradicated and preparing and using an mRNA therefor, whereby the treatment targets are drastically expanded.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the present invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the present invention and does not pose a limitation on the scope of the present invention unless otherwise claimed. No language herein should be construed as indicating any non-claimed element as essential to the practice of the present invention.

Preferred embodiments of the present invention are described herein, including the best mode known to the inventors for carrying out the present invention. Variations of those preferred embodiments may become apparent to those of ordinary skill in the art upon reading the foregoing description. The inventors expect skilled artisans to employ such variations as appropriate, and the inventors intend for the present invention to be practiced otherwise than as specifically described herein. Accordingly, the present invention includes all modifications and equivalents of the subject matter recited in the claims appended hereto as permitted by applicable law. Moreover, any combination of the above-described elements in all possible variations thereof is encompassed by the present invention unless otherwise indicated herein or otherwise clearly contradicted by context.

1. A method of preparing an artificial adjuvant vector cell (aAVC) co-expressing a target antigen and CD1d and having an ability to activate immunity against the target antigen comprising treating the target antigen and CD1d co-expressing cell with a CD1d ligand in a culture medium.

2. The method according to claim 1, wherein the target antigen is a tumor antigen or a pathogenic antigen.

3. The method according to claim 1, wherein the ability to activate immunity is against a tumor cell, virus or virus-infected cell.

4. The method according to claim 1, wherein the CD1d co-expressing cell is produced by the step of (a) transfecting a nucleic acid encoding a target antigen or CD1d into a cell, or (b) transfecting one or two molecular species of a nucleic acid encoding a target antigen and CD1d into a cell.

5. The method according to claim 1, wherein the nucleic acid is mRNA or a vector.

6. The method according to claim 5, wherein the cell is a syngeneic cell or allogeneic cell for a subject to which the cell is to be administered.

7. The method according to claim 5, wherein the cell is selected from;
(i) a CD1d-expressing cell;
(ii) a target antigen-expressing cell; or
(iii) a cell having no expressions of a target antigen and CD1d.

8. The method according to claim 7, wherein the CD1d-expressing cell is a CD1d transfectant.

9. The method according to claim 7, wherein the target antigen-expressing cell is a target antigen transfectant.

10. An artificial adjuvant vector cell (aAVC) co-expressing a target antigen and CD1d and having an ability to activate immunity against the target antigen, which is obtained by the method of claim 1.

11. The aAVC according to claim 10, wherein the target antigen is a tumor antigen or pathogenic antigen.

12. The aAVC according to claim 11, wherein the ability to activate immunity is against a tumor cell, virus or virus-infected cell.

13. The aAVC according to claim 1, wherein the CD1d co-expressing cell is produced by the step of (a) transfecting a nucleic acid encoding a target antigen or CD1d into a cell, or (b) transfecting one or two molecular species of a nucleic acid encoding a target antigen and CD into a cell.

14. The aAVC according to claim 13, wherein the nucleic acid is mRNA or a vector.

15. The method according to claim 5, wherein the cell is selected from;
(i) a CD1d-expressing cell;
(ii) a target antigen-expressing cell; or
(iii) a cell having no expressions of a target antigen and CD1d.

16. The method according to claim 15, wherein the CD1d-expressing cell is a CD1d transfectant.

17. The method according to claim 15, wherein the target antigen-expressing cell is a target antigen transfectant.

18. A composition comprising the aAVC according to claim 10.

19. The composition according to claim 19, further comprising an adjuvant.

20. An immunoinducer comprising the aAVC according to claim 10.

21. The immunoinducer according to claim 20, further comprising an adjuvant.

22. A method of inducing immunity comprising administering an effective amount of the aAVC according to claim 6 to a subject in need thereof, wherein the ability to activate immunity is against a tumor cell, virus or virus-infected cell.

23. The method according to claim 15, further comprising administering an adjuvant.

24. A kit comprising any of (1) to (8) below:
(1) a combination of (1-1) a CD1d-expressing cell, and (1-2) a construct for in vitro transcription of a nucleic acid encoding the target antigen;
(2) a combination of (2-1) a CD1d-expressing cell, and (2-2) a nucleic acid encoding the target antigen;
(3) a combination of (3-1) a construct for in vitro transcription of a nucleic acid encoding CD1d, (3-2) a construct for in vitro transcription of a nucleic acid encoding the target antigen, and (3-3) a cell that is allogeneic to a target in need of immunity induction;
(4) a combination of (4-1) a construct for in vitro transcription of a nucleic acid encoding CD1d and a nucleic acid encoding the target antigen, and (4-2) a cell that is allogeneic to a target in need of immunity induction;
(5) a combination of (5-1) a construct for in vitro transcription of a nucleic acid encoding CD1d, (5-2) a nucleic acid encoding the target antigen, and (5-3) a cell that is allogeneic to a target in need of immunity induction;
(6) a combination of (6-1) a nucleic acid encoding CD1d and the target antigen, (6-2) a construct for in vitro transcription of a nucleic acid encoding the target antigen, and (6-3) a cell that is allogeneic to a target in need of immunity induction;
(7) a combination of (7-1) a nucleic acid encoding CD1d and the target antigen, (7-2) a nucleic acid encoding the target antigen, and (7-3) a cell that is allogeneic to a target in need of immunity induction; and
(8) a combination of (8-1) a nucleic acid encoding CD1d and the target antigen, and (8-2) a cell that is allogeneic to a target in need of immunity induction.

25. The kit according to claim 14, further comprising a CD1d ligand.
